(19) United States
(12) Patent Application Publication
(10) Pub. No.: US 2002/0034537 A1
(43) Pub. Date: Mar. 21, 2002

(54) Cationic diagnostic, imaging and therapeutic agents associated with activated vascular sites

(76) Inventors: Brita Schulze, Walchensee (DE); Birgitta Sauer, Penzberg (DE); Marc Dellian, Munich (DE); Uwe Michaelis, Weilheim (DE); Michael Teifel, Penzberg (DE); Kurt W. Naujoks, Penzberg (DE); Claudia Biro, Muehldorf (DE)

Correspondence Address: MORGAN, LEWIS & BOCKIUS, 1800 M STREET NW, WASHINGTON, DC, 20036-5869, US

(21) Appl. No.: 09/847,538

(22) Filed: May 3, 2001

Related U.S. Application Data
(60) Provisional application No. 60/201,673, filed on May 3, 2000.

(51) Int. Cl. A61K 39/395; A61K 9/127

(52) U.S. Cl. 424/450; 424/155.1

(57) ABSTRACT

Methods and associated compositions are described for enhancing the selective delivery of therapeutic, diagnostic and imaging agents to activated vascular sites.

### RELATED APPLICATIONS

This application claims the benefit of U.S. provisional application no. 60/201,673, filed May 3, 2000, all of which is incorporated herein by reference in its entirety.

### FIELD OF THE INVENTION

The present invention relates to compositions and methods for preferentially targeting therapeutic, diagnostic and imaging agents to accumulate in the vicinity of activated vascular sites. Specifically, the present invention relates to compositions and methods that selectively target such agents to vascular endothelial sites where anionic charges are exposed or clustered at sites of angiogenesis or inflammation. More specifically, the present invention relates to compositions and methods useful in the treatment of diseases associated with angiogenesis such as cancer, diabetic retinopathy and retrolenta fibroplasia. In addition, the present invention relates to the modification and packaging of diagnostic, imaging and therapeutic agents to enhance their efficacy in connection with activated vascular sites as are associated with angiogenesis associated diseases and with the wound healing process. The invention relates also to modifications of drug carrier systems that can be adjusted such as to maximize their targeting effect while minimizing toxic side effects.

### BACKGROUND

The present invention relates to the discovery that activated vascular sites are associated with an enhanced negative charge relative to vascular endothelial cells in their quiescent state. In fact, the enhanced negative surface charge of activated endothelial cells and their associated extracellular matrix layer may function as a natural barrier against the penetration of negatively charged compounds from the blood.

1. Vascular Structure and Permeability.

Blood vessels, which enclose blood within the circulatory system and separate blood from tissues and extravascular fluid of the body, are lined by vascular endothelial cells in their luminal layer. Capillaries, the smallest blood vessels, are thin-walled microscopic vessels composed of a single layer of vascular endothelial cells. The walls of the capillaries are responsible for exchange of nutrients and metabolites and for the establishment and maintenance of fluid equilibrium between the intravascular and extravascular fluid compartments. Although lipophilic and small-molecular-weight hydrophilic molecules diffuse through these walls easily, they are generally impermeable to macromolecules. The vascular endothelial cells are connected to each other at tight junctions and, thus, provide a barrier to protect organs from uncontrolled exchange of molecules.

The blood vessel membranes composed of endothelial cells conntected by tight junctions are not impermeable. For example, a large number of macromolecules such as antibodies, protein-bound hormones, cytokines have access to the interstitial space and are ultimately returned to the plasma via the lymphatic system. In the early 1950's, Pappenheimer et al. suggested that pores having a radius of approximately 40 Å are present in capillaries to enable diffusion of small hydrophilic solutes (Rippe et al., 1994). Later, Grotte et al reported the presence of large pores of 250 Å to 300 Å for transcapillary passage of plasma proteins (Rippe et al., 1994). Over the years, the presence of pores and capillary selectivity based on size have been amply confirmed in numerous tissues (Rippe et al., 1994).

The brain is protected by the blood-brain barrier, which presents a relatively increased local negative charge on associated endothelial cells and their adjacent extracellular matrix. For example, Taguchi et al.(1998) showed that in the choroid plexus of the rat brain ventricles, the luminal surface and fenestral diaphragm of the capillary endothelium as well as its basement membranes and epithelium are strongly anionic. Taguchi et al (1998) also suggested that the negatively charged endothelial fenestrae and basement membranes may act as a charge barrier to inhibit the passage of anionic molecules.

Accordingly, the physicochemical properties of a molecule such as its charge, size, configuration, and polarity are understood to affect its transport across a blood vessel wall (Seno, 1983; Yuan, 1998). In general, the vascular permeability of a molecule is inversely correlated with its size. Additionally, the vessel walls are relatively more permeable to cationic than to anionic molecules, presumably because the basement membrane and the glycocalyx on the luminal surface of the vessel walls are negatively charged (Yuan, 1998). Consistent with such findings, Adamson et al. (1988) reported that the vascular permeability of ribonuclease (net charge +4; MW 13,683) is twice as high as that of a-lactalbumin, a molecule of similar size (MW 14,176) but is negatively charged (net charge -10).

While assessing the ontogeny of the microvascular endothelial barrier to anionic macromolecules, Henry et al. (1996) found that as the chicken chorioallantoic membrane ages, endothelial anionic sites became reduced. Henry et al. saw continuous cationic ferritin binding on the luminal endothelium, the junctional clefts, and the plasmalemma vesicles from days 4.5 to 14, but on day 18, the binding became discontinuous. Cavallo et al. (1980) studied surface charge characteristics of small blood vessels and perivascular components in rat cremaster vessels exposed to serotonin or mild thermal injury and discovered that leaky vessels showed increased density of anionic sites on the luminal endothelial plasma membrane.

2. Enhanced Negative Charge at Angiogenic and Active Vascular Sites.

Angiogenesis is the process by which new blood vessels are formed (Folkman et al., 1992). It is essential for normal body activities such as reproduction, development, and wound repair. Although the entire process of angiogenesis is not completely understood, it is believed that the process involves a complex set of molecules that interact with each other to regulate the growth of endothelial cells, the primary cells of capillary blood vessels. Under normal conditions, these molecules maintain the cells in quiescent state, i.e., a state of no capillary growth, for prolonged periods of time that may last for as long as weeks or, in some cases, decades. However, when necessary, such as during wound healing, these molecules will promote rapid proliferation and turnover of the cells within a five day period (Folkman et al., 1992; Folkman et al., 1987).

Although angiogenesis is a highly regulated process under normal conditions, many diseases are characterized by persistent unregulated angiogenesis. For example, ocular neovascularization has been implicated as the most common cause of blindness. In conditions such as arthritis, newly formed capillary blood vessels invade the joints and destroy cartilage. In diabetes, new capillaries formed in the retina invade the vitreous, bleed, and cause blindness. Growth and metastasis of solid tumors are also dependent on angiogenesis (Folkman et al. 1986; Folkman et al., 1989). Tumors which enlarge to greater than 2 mm must obtain their own blood supply and do so by inducing the growth of new capillary blood vessels. These new blood vessels embedded within the tumor provide a means for tumor cells to enter the circulation and metastasize to distant sites such as liver, lung, or bone (Weidner et al., 1991).

Vascular leakiness in tumors is in general higher than in normal tissue (Yuan et al 1994). Yuan et al. (1995) showed that tumor vessels are more permeable than normal vessels due to the presence of large pores of about 400 nm in diameter in the vessel walls. It is thought that the leakiness during angiogenesis of normal tissue and tumors is a consequence of endothelial cells relaxing and loosening their tight junction in order to divide and multiply. Alternatively, vascular leakiness has been suggested to be required for angiogenesis to proceed (Dvorak et al., 1995).

However, in what may be a homeostatic response to such leakiness, the angiogenic endothelial cells at active vascular sites appear to express a greater density and/or higher amount of negatively charged surface molecules than are found in the quiescent state at vascular sites. This finding is supported by the work of authors such as Cavallo et al. (1980) who showed that leaky vessels showed increased density of anionic sites on the luminal endothelial plasma membrane as compared to controls. This enhancement of negative charge functions as a natural barrier against the penetration of negatively charged molecules from the blood system. As noted above, Taguchi et al. (1998) showed that increased negative charge at the endothelial fenestrae and basement membranes may act as a charge barrier to inhibit the passage of anionic molecules.

Cationic liposomes have been demonstrated to be taken up by endothelial cells in an organ specific pattern with highest accumulation in the lung (McLean et al., 1997). However, angiogenic endothelial cells of tumors and in chronic inflammation revealed a preferential uptake of cationic liposomes, with a high proportion being associated with endothelial fenestrae (Thurston et al., 1998). Endothelial fenestrae are very frequently found on tumor endothelium (Roberts & Palade, 1997; Hobbs et al., 1998), and may thus be the site of extravasation of cationic proteins.

3. Targeting of Angiogenic Endothelial Cells with Cationic Liposomes

McDonald et al., U.S. Pat. No. 5,322,678 (1998), describes selectively targeting angiogenic endothelial cells using cationic liposomes containing an agent that affects the growth of the target cells or that labels the target cells. The cationic liposomes associate with angiogenic endothelial cells for a sufficient period of time and in a manner such that the liposomes themselves and/or the contents of the liposomes enters the angiogenic endothelial cells. Thus, the agent that enters the cell can inhibit or promote angiogenesis of the cell or merely provide a label allowing detection of the site of angiogenesis. The invention of McDonald et al. is based on the discovery that cationic liposomes associate with angiogenic endothelial cells at a five fold or greater ratio than they associate with corresponding, quiescent endothelial cells.

This McDonald et al. patent describes the use of cationic liposomes that may include both neutral and cationic lipids, for example, having 5 mol % or more of cationic lipids or, specifically, having neutral lipids in an amount of about 45% and cationic lipids in an amount of about 55%. While McDonald et al. indicates that cationic liposomes have a zeta potential of greater than 0 mV, this patent does not teach any specific zeta potential or isoelectric point, or ranges thereof, as being preferred for the selective targeting of angiogenic endothelial cells. McDonald et al. also does not indicate the preferred upper limit of cationic lipid to use in the cationic liposome composition for selective targeting of angiogenic endothelial cells. 100161 Thurston et al. (1998) also describes the targeting of endothelial cells in tumors and chronic inflammation in mice using cationic liposomes. Thurston uses cationic liposomes having 55 mol % of cationic component. Thurston does not disclose the specific zeta potential or isoelectric point or ranges thereof for selective targeting of endothelial cells in tumors or chronic inflammation in mice.

4. Modulation of Protein Pharmacokinetics.

Morgan et al., U.S. Pat. No. 5,322,678 (1994) and U.S. Pat. No. 5,635,180 (1997), disclose a method of altering the pharmacokinetics of proteins by modifying their charge. These patents describe methods to modulate the renal clearance of “targeting proteins,” particularly antibody fragments, based on a finding that the more cationic antibody or fragments are more readily deposited in the glomerular basement membrane and thus are more rapidly cleared from the serum. Thus, according to Morgan et al., the net charge of protein agents may be altered either to increase or decrease their rate of clearance from the bloodstream.

The Morgan et al. patents observe that tumor cells have a net negative surface charge and that normal cells similarly have clusters of negative charge. Notwithstanding the negative charge of tumor cells, Morgan teaches that the charge of a targeted therapeutic protein should be made more negative (that is, more anionic, or in other words, having an isoelectric point of less than 7) in order to reduce renal clearance, increase serum half life and minimize nonspecific interactions with normal cells. This is said to allow increased localization of the agent at a target site such as a tumor. Morgan et al. teaches modifying the therapeutic agent to have a more acidic isoelectric point with an acid shift of at least one-tenth of a pH unit and no more than four pH units and more preferably, an isoelectric point acidic shift of approximately one pH unit. Morgan et al. explains that at a pI of 4.0 or lower (more acid), virtually all ionizable groups are protonated in the protein. One of the ways to accomplish this is by charge modifying at least one lysine residue in the targeting protein from a net positive charge to a net negative charge. See, e.g., U.S. Pat. No. 5,635,180 at cols. 6-8 and 11 and U.S. Pat. No. 5,322,678 at cols. 6-8.

In contrast, for diagnostic imaging products, Morgan et al. teach that a neutral or basic antibody fragment is needed for increased serum clearance and for high tumor-to-background ratios at early time points. However, relatively more basic (cationic) fragments are said to be preferred for short half-life isotopes while relatively more acidic (anionic) fragments are preferred for isotopes having longer half lives. Accordingly, Morgan et al. teaches a more basic isoelectric point for diagnostic agents and a more acidic isoelectric point for therapeutic agents. Nevertheless, Morgan et al. does not provide guidance for the adjustment of charge or isoelectric point of either the therapeutic or diagnostic agent for maximum targeting and minimum toxicity.

In contrast to Morgan et al., Khawli et al., U.S. Pat. No. 5,990,286 (1999) describes the diagnostic use of antibodies with a reduced net positive charge (an acidic shift in isoelectric point) rather than an increased net positive charge. According to Khawli et al, this is in order to increase antigen binding specificity, decrease non-specific binding and decrease in vivo clearance time. Disclosed methods include the steps of obtaining an intact antibody having binding specificity for an antigen to be detected, the native antibody having a plurality of free amino groups disposed thereon, reacting at least one of the free amino groups with a chemical agent to produce a modified antibody, such that the modified antibody has an isoelectric point lower than the isoelectric point of the intact antibody, and labeling the modified antibody with a detectable label. The method is said to produce a labeled modified antibody that can be detectable, for example, by immunoscintigraphy, such as by a gamma camera.

Similar to Khawli et al., a paper by Rok et al. in Renal Failure 20 (2): 211-217 (1998) reported data that indicate that the excretion of a drug -LMWP conjugate into the urine can be increased by decreasing the positive charge (an acidic shift in isoelectric point) on the carrier surface. Thus, such a carrier was said to be an attractive candidate for drug targeting to the bladder.

These prior art teach the modification of the charge of therapeutic agents to decrease their net charge, that is to make such agents more anionic, in order to increase their localization to target site, unlike the present invention which is based in part on the surprising finding that positively charged therapeutic and diagnostic agents are selectively targeted to activated vascular sites which are negatively charged or show a clustering of anionic charges as compared to inactive vascular sites. The present invention teaches modifying therapeutic and diagnostic agents to increase their net zeta potential or isoelectric point for selective targeting to activated vascular sites. The present invention provides guidance for the range of zeta potentials and isoelectric points for selective targeting to activated vascular sites.

At the present, due to the lack of clinically proven methods for delivering therapeutic and diagnostic agents selectively to target sites, for example, sites of angiogenesis or inflammation, such conditions are treated directly by physical means such as surgical removal of cancerous tissues. Sites of angiogenesis also are treated by chemical means. For example, chemotherapeutic agents are applied to cancers and anti-inflammatory drugs are applied to treating chronic anti-inflammatory conditions. However, these treatments raise concerns with respect to operative risks, side effects, efficacy, and success rate. Further, treatment such as chemotherapy is not targeted and side effects such as bone marrow depression, gastroenteritis, nausea, alopecia, liver or lung damage, and sterility from chemotherapy can result. Thus, there exist a need for the development of novel strategies that will selectively deliver therapeutic and diagnostic agents to activated vascular sites as found in various angiogenesis-associated diseases.

The enhancement of negative charge at activated vascular sites, i.e., areas where angiogenesis is occurring, provides a means for distinguishing quiescent endothelial cells from activated cells. Such negatively charged, activated vascular sites can serve as targets for therapeutic and diagnostic agents modified to bear a net positive charge or a positive charge within the ranges described below. Therapeutic, imaging and diagnostic agents can be modified to bear a positive charge and targeted selectively to activated vascular sites.

### SUMMARY OF THE INVENTION

The present invention provides a method of selectively targeting a therapeutic, diagnostic or other pharmaceutical composition to an activated vascular site by modifying its charge or charge density, respectively. Closely correlated to such charge modification of a drug or drug carrier composition is a change in its tolerability. Positively charged drug carrier systems are often considered to be biologically poorly tolerable; the toxicity typically increases with the amount of positively charged component. As demonstrated in the examples, the inventors surprisingly have found that there is a linear relationship between the targeting behavior of a drug carrier system and its zeta potential. However, the relationship between zeta potential and cationic component concentration is best fitted by a hyperbolic curve. This allows for identification of a region where the targeting is almost at its maximum but the cationic component concentration is not. This method of selectively targeting preferably is practiced by the administration of a composition selected from the group consisting of: (a) particles, excluding liposomes, having a zeta potential in the range of about +25 mV to +100 mV in about 0.05 mM KCl solution at about pH 7.5; (b) molecules having an isoelectric point above 7.5; and (c) liposomes containing cationic lipids in the range of about 25 mol % to 50 mol %; (d) magnetosomes with a cationic lipid layer having a zeta potential in the range of about +25 mV to +100 mV in about 0.05 mM KCl solution at about pH 7.5; (e) oil-in-water emulsions or microemulsions containing cationic amphiphiles in the outer layer in the range of about 25 to 60 mol % or having a zeta potential of about +25 mV to +100 mV in about 0.05 mM KCl solution at about pH 7.5. The contemplated activated vascular sites include: (a) sites of angiogenesis; (b) sites of inflammation; (c) sites of wound healing; and (d) the blood brain barrier, and other such sites will be apparent to persons skilled in the art.

Preferably, an imaging composition for selective targeting to an activated vascular site would include an imaging agent and a carrier. A therapeutic composition for selective targeting to an activated vascular site would include a therapeutically effective amount of an active ingredient and a carrier and possibly an imaging agent as well. Contemplated carriers include: (a) particles, excluding liposomes, having a zeta potential in the range of about +25 mV to +100 mV in about 0.05 mM KCl solution at about pH 7.5; (b) molecules having an isoelectric point above 7.5; and (c) liposomes containing cationic lipids in the range of about 25 mol % to 50 mol %; (d) magnestomes with a cationic lipid layer having a zeta potential in the range of about +25 mV to +100 mV; (e) oil-in-water emulsions or microemulsions containing cationic amphiphiles in the outer layer in the range of about 25 to 60 mol % or having a zeta potential in the range of +25 mV to +100 mV in about 0.05 mM KCl solution at about pH 7.5. Appropriate imaging agents include iron oxide particles, dyes, fluorescent dyes, NMR labels, scintigraphic labels, gold particles, PET labels, ultrasound contrast media, and CT contrast media.

Therapeutic, diagnostic and imaging methods used with animals, including mammals and particularly human beings, involve the administration of agents in a protocol that permits the agent or active ingredient to selectively accumulate to a effective level for imaging or other diagnostic purposes in the vicinity of the site of angiogenesis.

Contemplated routes of administration include oral administration, intravenous administration, transdermal administration, subcutaneous administration, intraperitoneal administration, intratumoral administration, intraarterial administration, and intramuscular administration, instillation and aerosol administration.

In a preferred embodiment, the active ingredient of a therapeutic composition would be selected from the group consisting of cytostatics and cytotoxic agents. Examples of cytostatics and cytotoxic agents include, but are not limited to, taxanes, inorganic complexes, mitose inhibitors, hormones, anthracyclines, antibodies, topoisomerase inhibitors, antiinflammtory agents, alkaloids, interleukins, cytokines, growth factors, proteins, peptides, tetracyclines, and nucleoside analogs. Specific examples of such agents, include but not limited to, paclitaxel and derivatives thereof, docetaxel, and derivatives thereof, epothilon A, B, D and derivatives thereof, camptotecin, daunorubicin, doxorubicin, epirubicin, vincristine, navelbine, antimicrotubuli active agents, thrombospondin, angiostatin, cis-platinum compounds and other platinum compounds, gemcitabine, and 5′-fluorouacil and other nucleoside analogs.

Another aspect of the present invention involves the modification of various compositions to have one or more of the characteristics selected from the group consisting of: (a) a zeta potential in the range of about +25 mV to +100 mV in about 0.05 mM KCl solution at about pH 7.5; and (b) an isoelectric point above 7.5.

It is contemplated that a variety of diseases may be treated with the foregoing methods and compositions. Such diseases include, for example, diabetic retinopathy, chronic inflammatory diseases, rheumatoid arthritis, inflammation, dermatitis, psoriasis, stomach ulcers, hematogenous and solid tumors. In some instances, the identification of an activated vascular site will be indicative of an angiogenesis associated disease.

It is contemplated that either the active agent or the carrier may be modified so that the zeta potential of the combined product is increased or decreased in order to achieve a zeta potential within the preferred zeta potential ranges. Such modifications may be achieved by chemical methods known to persons skilled in the art, and preferably involve cation forming reagents and/or cationic reagents such as but not limited to ethylene diamine, hexamethylenediamine, triethylene tetraamine, 4-dimethylamino butylamine, N,N-dimethylaminoethyl amine, other cationic polyamines, dimethylamino benzaldehyde, polylysine, other cationic peptides, chitosan and other cationic polysaccharides. Such modifications can also be achieved by reaction of the active agent with a cationic molecule resulting in a covalent bond between the two molecules. Alternatively, such modifications can be achieved by complexation (i.e., formation of a noncovalent bond) of the active agent with a cationic agent or carrier system. Some compositions may comprise a protein, and various reagents to increase or decrease zeta potential will be known to protein and medicinal chemists.

In a preferred embodiment of the present invention, the diagnostic, imaging and therapeutic compositions will be labeled or packaged with directions for the administration of the composition to treat an angiogenesis associated disease.

It is contemplated that the active ingredient in the therapeutic composition is selected from the group consisting of etherlipid, alkyllysolecithin, alkyllysophopholipid, lysolipid, alkylphospholipid. Preferably, etherlipid in the composition includes but are not limited to 1-O-octadecyl-2-O-methyl-rac-glycero-3-phosphocholine, 1-O-Hexadecyl-2-O-methyl-sn-glycerol, Hexadecyl phosphocholine, and Octadecylphosphocholine.

In a preferred embodiment the therapeutic composition is effective to inhibit inflammation, to promote bone repair, or to promote wound healing.

Preferably, zeta potential of compositions administered would fall within the range of about +25 mV to +60 mV in about 0.05 mM KCl solution at about pH 7.5, more preferably with a range of about +30 to +50 mV in about 0.05 mM KCl solution at about pH 7.5. In another embodiment of the invention, known diagnostic, imaging and therapeutic compositions are modified, either to increase or decrease the composition's effective zeta potential so that it falls within the preferred ranges described above or in the detailed description, such as within a broad range of about +25 mV to +60 mV in about 0.05 mM KCl solution at about pH 7.5.

Various cationic lipids in addition to DOTAP are contemplated by the present invention. Examples include, but are not limited to, DDAB (dioctadecyl-dimethyl-ammoniumbromide), DC-Chol (3β[N-(N′,N′-dimethylaminoethane)-carbamoyl)] cholesterol, DOSPER (1,3-dioleoyl-2-(6-carboxy-spermyl)-propyl-amid).

The present invention provides a method of determining an optimal range of zeta potential for a composition for targeting to a specific site. The method comprising i) measuring the zeta potential of the composition while varying concentration of cationic components; ii) plotting the values of zeta potential on the y axis and the concentrations of cationic components on the x axis to obtain a hyperbolic curve; and iii) determining zeta potential and concentration of cationic component in the region where the hyperbolic curve inflects, wherein the region of inflection of the hyperbolic curve provides an optimal range of zeta potential for the composition. The present invention provides a method for identifying an optimal range of zeta potential for a composition for targeting to a specific site comprising evaluating zeta potential for the composition, wherein the composition is associated with different amounts of a cationic component, and identifying an optimal range of zeta potential. The present invention also provides a method of modifying a composition to enhance its efficacy comprising the associating of cationic components with the composition to produce a composition having an optimal range of zeta potential.

### BRIEF DESCRIPTION OF THE DRAWINGS

FIG. 1 shows a schematic representation of a particle's zeta potential.

FIG. 2 shows the measured zeta potentials for various liposomal formulations.

FIG. 3 shows the zeta potential of cationic liposomes fitted to a hyperbolic curve with zeta potential dependent on DOTAP (1,2-dioleoly-3-trimethylammonium propane) concentration in mol %.

FIGS. 4A-C shows the selectivity of neutral, negative and positive charged dextrans over time in terms of the relative fluorescence intensities in tumor endothelial cells versus their surrounding tissue.

FIGS. 5A-B show the uptake of rhodamine-labeled liposomes by HUVEC.

FIG. 5A shows fluorescence intensity (cps) vs. DOTAP (mole %).

FIG. 5B shows fluorescence intensity (cps) vs. zeta potential (mV).

### DETAILED DESCRIPTION OF THE INVENTION

The present invention is based on a discovery that molecules having a specified net positive range or charge can be selectively targeted to activated vascular sites. Such sites are found in association with angiogenic endothelial cells, sites of inflammation and sites of would healing.

The present invention also is based on a discovery that increasing or modulating the net positive charge of diagnostic, imaging and therapeutic agents results in the selective accumulation of such agents at activated vascular sites. Moreover, by selecting particular charge ranges or charge densities, the accumulation of such agents at sites of inflammation, as are chronically found in the lung, may be minimized while providing targeting selectivity as between activated and nonactivated vascular sites in other tissues. In this regard, it is contemplated that agents having a net positive charge below the ranges indicated below may be treated, modified or packaged so as to increase their apparent net charge. It also is contemplated that agents having a net positive charge in excess of the preferred ranges described below may be treated, modified or packaged so as to decrease their apparent net charge to improve their biological tolerability.

In addition, the present invention is based on a discovery that molecules having a specified net positive charge selectively bind to and are taken up by angiogenic endothelial cells relative to both quiescent endothelial cells and to endothelial cells at relative constant activated vascular sites such as the lung. Such selective targeting and accumulation increases the local binding of these agents to the extracellular matrix and to angiogenic endothelial cells. In addition, this selective targeting and local accumulation of agents occurs in the vicinity of vascular endothelial cells present at activated vascular sites associated with inflammation as distinguished from sites of neovascularization induced by tumors. Such accumulation, in either type of activated vascular site, also produces a higher concentration gradient of these agents at the sites of inflammation or metastatic tumors. Through extravasation and other relevant processes, such agents selectively accumulate at such target sites for therapeutic, imaging and diagnostic purposes.

Accordingly, the present invention provides a method of selectively targeting a diagnostic, imaging or therapeutic agent to activated vascular sites of a mammal, including human patients. In general, the invention involves the administration of agents having a net positive zeta potential above 25 mV in about 0.05 mM KCl at about pH 7.5 or isoelectric point above 7.5, preferably in the ranges described below, and allowing the agent to selectively accumulate at one or more activated vascular sites.

Such agents can be targeted to the vascular endothelial cells found at sites of inflammation and to angiogenic endothelial cells and their extracellular matrix for a time and in a manner such that the agent accumulates in the vicinity of the targeted vascular endothelial cells. The present invention also provides agents comprising a carrier having an specified net positive zeta potential above 25 mV in about 0.05 mM KCl at about pH 7.5 or isoelectric point above 7.5, in the ranges provided below, and an active ingredient. In addition, the compositions and methods of the present invention may be used at activated vascular sites associated with wound healing where the junctions between vascular endothelial cells have become leaky and the cells and their extracellular matrices have developed an increased negative charge relative to quiescent or non-activated vascular sites.

In this regard, a person skilled in the relevant art would understand that zeta potential is a measurement applicable to the charge or charge density of particles, particularly to colloidal particles such as liposomes, magnetosomes or microemulsions larger than about 3 to 10 nm in size. Similarly, it would be understood that isoelectric point is a measurement applicable to the charge density of macromolecules including proteins, antibodies, and colloids, such as dextrans.

1. Definitions.

Unless defined otherwise, all technical and scientific terms used in this specification shall have the same meaning as commonly understood by persons of ordinary skill in the art to which the present invention pertains.

“Activated vascular site” refers to vascular endothelial sites exhibiting an activated phenotype and where the tight junctions normally found between endothelial cells may be loosened as a result of angiogenesis or inflammation, and the permeability of this site increases, permitting extravasation of blood, plasma and various pharmaceutical agents.

“Active ingredient” refers to an agent that is diagnostically or therapeutically effective.

“Angiogenesis” refers to the formation of new blood vessels. Endothelial cells form new capillaries in vivo when induced to do so, such as during wound repair or in tumor formation or certain other pathological conditions referred to herein as angiogenesis-associated diseases.

The term “angiogenesis-associated disease” refers to certain pathological processes in humans where angiogenesis is abnormally prolonged or pathologically induced. Such angiogenesis-associated diseases include diabetic retinopathy, chronic inflammatory diseases, rheumatoid arthritis, dermatitis, psoriasis, stomach ulcers, hematogenous tumors, and other types of human solid tumors.

“Angiogenic endothelial cells” refers to vascular endothelial cells undergoing angiogenesis that are proliferating at a rate substantially higher than the normal proliferation rate for vascular endothelial cells in general.

“Carrier” generally refers to a diluent, adjuvant, excipient, or vehicle with which a diagnostic, imaging or therapeutic is administered. As used herein, the term carrier also refers to a pharmaceutically acceptable component(s) that contains, complexes with or is otherwise associated with an active ingredient in order to facilitate the transport of such an agent to its intended target site. Contemplated carriers include those known in the art, liposomes, various polymers, lipid complexes, serum albumin, antibodies, cyclodextrins, and dextrans, chelates and other supramolecular assemblies.

“Cationic” refers to an agent that has a net positive charge or positive zeta potential (or, an isoelectric point above 7) at physiologic pH.

“Colloids” or colloidal particles are particles dispersed in a medium in which they are insoluble, and having a size between 10 nm and 5000 mn.

“Combination” or “co-administration” refers to an administration schedule that is synchronous, serial, overlapping, alternating, parallel, or any other treatment schedule in which the various agents or therapies are administered as part of a single treatment regimen, prescription or indication or in which the time periods during which the various agents or therapies that are administered otherwise partially or completely coincide.

“Diagnostic or imaging agent” refers to a pharmaceutically acceptable agent that can be used to localize or visualize site of angiogenesis by various methods of detection, including MRI and scintigraphic techniques. Contemplated diagnostic or imaging agents include those known in the art, such as dyes, fluorescent dyes, gold particles, iron oxide particles and other contrast agents including paramagnetic molecules, x-ray attenuating compounds (for CT and x-ray) contrast agents for ultrasound, y-ray emitting isotopes (Scintigraphy), and positron-emitting isotopes (PET).

“Diagnostically effective” refers to an agent that is effective to localize or otherwise identify a site of angiogenesis or neovascularization for monitoring or imaging purposes.

“Emulsion” or “microemulsion” refers to a system containing two immiscible liquids in which one is dispersed, in the form of very small globules (internal phase) throughout the other (external phase), for example, oil in water (milk) or water in oil (mayonnaise). Emulsion or microemulsion can be a colloidal dispersion of two immiscible liquids (e.g., a liquid-liquid dispersion).

“Endothelial cells” refers to those cells making up the endothelium, which is the monolayer of cells that line the inner surface of the blood vessels, the heart, and the lymphatic vessels. These cells retain a capacity for cell division, although they proliferate very slowly under normal (that is, non-angiogenic) conditions, undergoing cell division only about once a year.

“Highly toxic” or “highly toxic agent” refers to a protein or peptide that is expressed in a target cell and inhibits the synthesis of protein, DNA or RNA, or destabilizes the lipid surface, or otherwise results in cell death by apoptosis or necrosis. Such agents are described in related application Ser. No. 60/163,250 filed Nov. 3, 1999.

“Increasing the zeta potential” or “increasing the isoelectric point” refers to a change or modification in an active ingredient or a carrier compound to increase its net positive charge by an amount that results in a statistically significant change in the rate or amount of accumulation of that ingredient or carrier at an activated vascular site, such as a site of angiogenesis, as would be achieved by derivatization, covalent modification, substitution or addition of amino acids, complexing or attachment to carrier or other substrate, relative to the accumulation of that ingredient or carrier prior to such change or modification.

“Isoelectric point” (pI or IEP) refers to the pH at which a molecule carries no net charge.

“Magnetosomes” also called ferrosomes, refers to an about nanometer-sized magnetite core enwrapped by one or more lipid layers.

“Oil-in-water emulsion” is a dispersion of colloidal droplets of hydrophobic oil coated by a layer of amphiphilic lipids in aqueous medium.

“Selectively target” or “selectively associate” with reference to an activated vascular site, such as an angiogenic capillary vessel, refers to the accumulation of an agent in the vicinity of, or the binding and/or uptake of an agent to angiogenic endothelial cells or their extracellular matrix at a higher level than would be found with corresponding normal (i.e., nonangiogenic) endothelial cells.

“Selectivity” with reference to fluorescence intensity refers to the ratio of relative fluorescence intensity of tumor endothelial cells to fluorescence intensity of surrounding tissue. Thus, in Example 5, selectivity is measured as a value for the affinity with which charged dextran molecules bind to the tumor endothelium.

“Therapeutically effective” refers to an agent that is effective to reduce the amount or extent of the pathology of an inflammatory disease or an angiogenesis associated disease, such as cancer, or to reduce the rate of the process of angiogenesis or neovascularization, preferably to substantially prevent the continuation of such processes at existing sites of angiogenesis, or to substantially prevent the initiation of angiogenesis at additional, undesirable sites of angiogenesis. For example, in the case of treating angiogenesis related to tumor metastasis, a therapeutically active or effective agent would show significant antitumor activity or tumor regression either through direct action upon tumor cells or through inhibition of angiogenesis. Such a compound might, for example, reduce primary tumor growth and, preferably, the metastatic potential of a cancer. Alternatively, such a compound might reduce tumor vascularity, for example either by decreasing microvessel size or number or by decreasing the blood vessel density ratio.

“Tumor regression” refers to a decrease in the overall size, diameter, cross section, mass or viability of a tumor; tumor marker reduction or a positive indication from other conventional indicia of cancer diagnosis and prognosis that indicates a reduction or growth slowing of cancer cells, as a result of the treatment of a cancer patient with compositions according to the present invention. Preferably, the administration of such compounds results in at least about a 30 percent to 50 percent tumor regression, more preferably at least about a 60 to 75 percent tumor regression, even more preferably at least about an 80 to 90 percent tumor regression and most preferably at least about a 95 or a 99 percent tumor regression at one or more tumor sites in a cancer patient. Ideally, such administration results in the killing or eradication of viable tumor cells or completely eradicates the tumor cells at one or more tumor sites in a cancer patient, leading to a clinically observable remission or other enhancement in health of a patient.

“Vicinity of a site of angiogenesis” refers to the physical proximity of an active ingredient to angiogenic endothelial cells and neovasculature such that a localized concentration gradient is achieved that is capable of delivering an amount of the active ingredient that is diagnostically or therapeutically effective with respect to an angiogenesis associated disease.

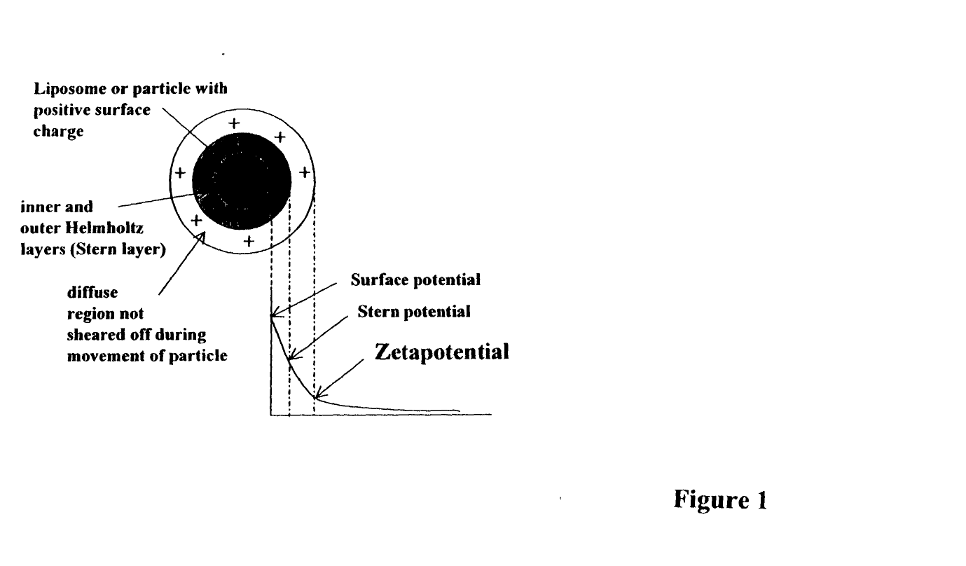

“Zeta potential” refers to measured electrical potential of a particle, such as a colloidal particle, measured with an instrument such as a Zetasizer 3000 using Laser Doppler micro-electrophoresis under the conditions specificed. The zeta potential describes the potential at the boundary between bulk solution and the region of hydrodynamic shear or diffuse layer (see FIG. 1). The term is synonymous with “electrokinetic potential” because it is the potential of the particles which acts outwardly and is responsible for the particle's electrokinetic behavior.

2. Detailed Description

A. Uptake of Charged Dextran Coated Iron Oxide Particles by HUVEC

The transport of macromolecules to endothelial cells is dependent not only on the size but also the charge of a molecule. For example, McDonald et al. (U.S. Pat. No. 5,837,283) discloses that cationic liposomes selectively target angiogenic endothelial cells that supply nutrients to a tumor. Moreover, Spragg et al. (1997) teaches that activated human umbilical vein endothelial cells (HUVEC) incubated with E-selectin-targeted immunoliposomes comprising the cytotoxic agent doxorubicin, exhibited significantly decreased cell survival, whereas unactivated HUVEC were unaffected by such treatment. The E-selectin-targeted immunoliposomes comprise cationic liposomes conjugated to a monoclonal antibody specific for E-selectin. E-selectin is an endothelial-specific cell surface molecule expressed at sites of activation in vivo and inducible in HUVEC by treatment with cytokines. It is known to the skilled artisan that the cell surface or the glycocalyx of the tumor endothelium is negatively charged.

The present invention is based in part on the discovery that in human umbilical vascular endothelial cell cultures, the uptake of iron oxide coated with positively charged dextran is greater than the uptake of iron oxide coated with neutral or negatively charged dextran. As shown in Table 1, when HUVEC were incubated with iron oxide coated with positively charged dextran, 51.8% of the iron oxide was taken up by the cells and found in the cell lysate, while 48.2% remained in the medium. On the other hand, when HUVEC were incubated with iron oxide coated with negatively charged dextran, 28.7% of the iron oxide was found in the cell lysate, while 71.3% remained in the medium. Moreover, when HUVEC were incubated with iron oxide coated with neutral dextran, 18.4% of the iron oxide was found in the cell lysate and 81.6% was found in the medium.

B. Correlation of Cationic Charge and Targeting

Although the prior art suggests that the transport of macromolecules, such as antibodies and liposomes, magnetosomes and microemulsions to a tumor site is dependent on the charge of the molecule, the prior art does not teach any particular range of zeta potential or positive charge to enhance the selective targeting of diagnostic, imaging and therapeutic agents to activated vascular sites, such as angiogenic endothelial cells and their extracellular matrix.

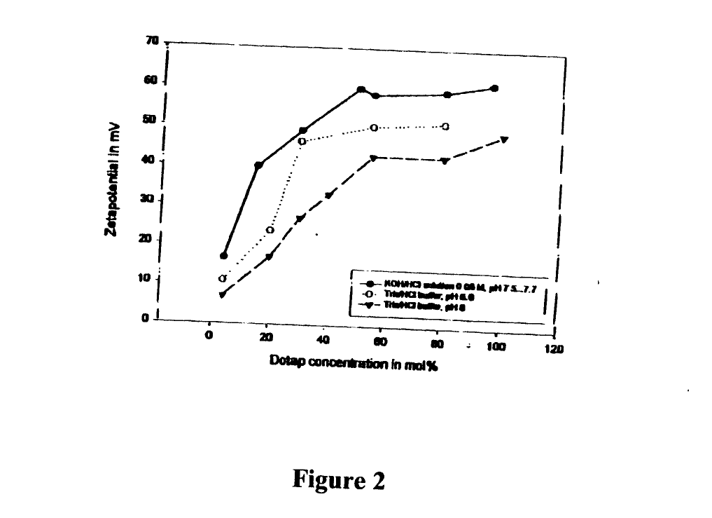

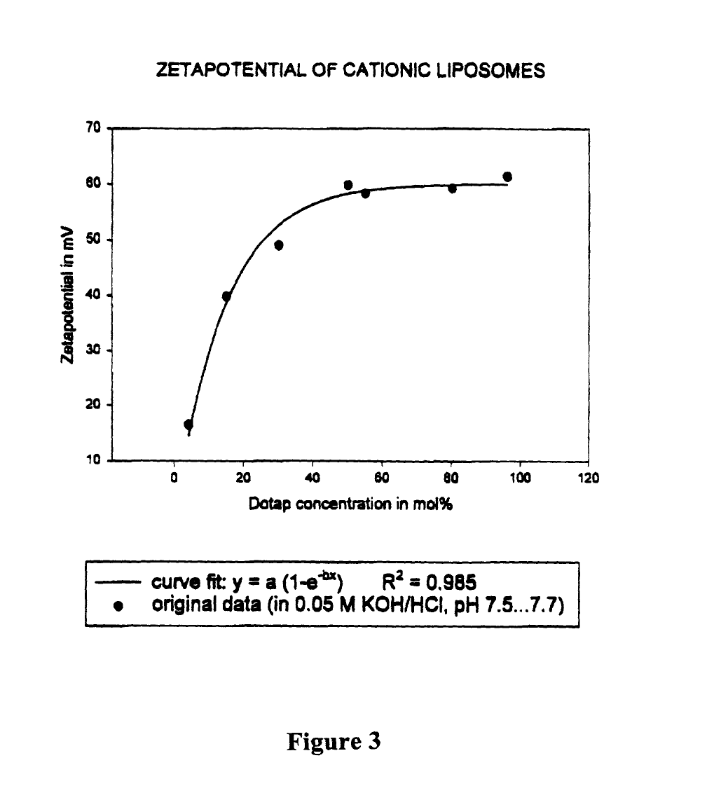

The present invention is based in part on the finding that increasing mol % of DOTAP ((1,2-Dioleoyl),sn-3-Glycerotrimethylammonium propane) or any other positively charged lipid (monovalently or polyvalently charged), as found in a cationic liposome, as well as in magnetosomes and oil-in-water microemulsions correlates with zeta potential of the macromolecule and its selective association in the vicinity of angiogenic endothelial cells. As shown in Tables 2 and 3, for DOTAP concentrations ranging between 4 and 50 mol %, there is a relatively constant increase in zeta potential. However, for DOTAP concentration of greater than 50 mol %, the zeta potential levels off and reaches a maximum of about +60 mV at pH 7-7.5. The graphs of the results of Tables 2 and 3 disclose hyperbolic curves (FIGS. 2 and 3) which are maintained even when the zeta potential is measured in different buffer systems. Although the absolute zeta potential changes slightly in a different buffer, the hyperbolic shape of the graph of the results is maintained.

The data indicate that the relationship between the net positive charge to the cationic component in a supramolecular assembly (e.g., liposome) is not linear (FIG. 2). Above DOTAP concentration of 60 mol %, the zeta potential ends in a plateau, i.e., further increases in the concentration of the cationic component does not increase the zeta potential. There seems to be a maximum of charge density beyond which further addition of cationic component has no advantage. Increasing the zeta potential of therapeutic, imaging or diagnostic agents beyond this zeta potential will likely increase non-specific binding at tissues other than the target site(s) and, thus, toxicity and other side effects. It may also increase the rate of clearance, thereby decreasing the effective dose available at the target site.

Based on these data, a preferred therapeutic, imaging or diagnostic agent of the present invention is formulated to optimize its selective association at an activated vascular site. However, as was surprisingly found by the present inventors, the zeta potential of such agents in particle form preferably should remain below the point at which any further increase in zeta potential no longer produces a corresponding increase in uptake by angiogenic endothelial cells or accumulation of such agents at activated vascular sites. In this way, the benefits of selective accumulation are achieved and the amount of nonspecific binding and side effects of such agents can be minimized.

Thus, for example, the diagnostic, imaging and therapeutic compositions according to the present invention preferably produce a net zeta potential in the range of about +25 to +100 mV in about 0.05 mM KCl at about pH 7.5 or have a cationic component in the range of about 20 to 60 mol % under the described conditions. Preferably, a range of about +25 to +60 mV in about 0.05 m.M KCl at about pH 7.5 or a cationic component of about 25 to 50 mol % is utilized. More preferably, a range of about +25 to +55 mV in about 0.05 mM KCl at about pH 7.5. Most preferably, a range of about +30 to +50 mV in about 0.05 mM KCl at about pH 7.5. Thus, for liposomes formulated with DOTAP and neutral lipids, the optimal amount of DOTAP is in the range of about 20 to 60 mol % and more preferably about 35 or 50 mol %.

In general, based on the results shown in FIGS. 2 and 3, it is preferable to have at least 25 mol % and at most 60 mol % cationic component. FIGS. 2 and 3 show that from 0 to 50 mol %, the corresponding zeta potential rises linearly. Below 25 mol %, the corresponding zeta potential is at the lower half of the curve. Therefore, targeting to angiogenic endothelial cells would not be appropriate for the purposes contemplated herein. Above 60 mol %, not much selective targeting is gained. Thus, 60 mol % of the cationic component is the preferred upper limit. The inflection point of the uptake curves also may be considered as providing optimal formulation. Preferably, the optimal region of the inflection of the curve is about +10 mV from the zeta potential at the inflection point or about +10 mol % from the concentration of the cationic component at the inflection point.

Whereas zeta potential applies to colloid particles, the same targeting behavior is observed for cationic molecules. In the present application, the preferred isoelectric point is above 7.5.

Examples of other cationic lipids that are contemplated by the present invention include, but not limited to, DDAB (dioctadecyl-dimethyl-ammoniumbromide), DC-Chol (3β[N-(N′,N′-dimethylaminoethane)-carbamoyl)] cholesterol, DOSPER (1,3-dioleoyl-2-(6-carboxy-spermyl)-propyl-amid).

C. Selective Targeting of Positively Charged Molecules to Angiogenic Endothelial Cells In Vivo

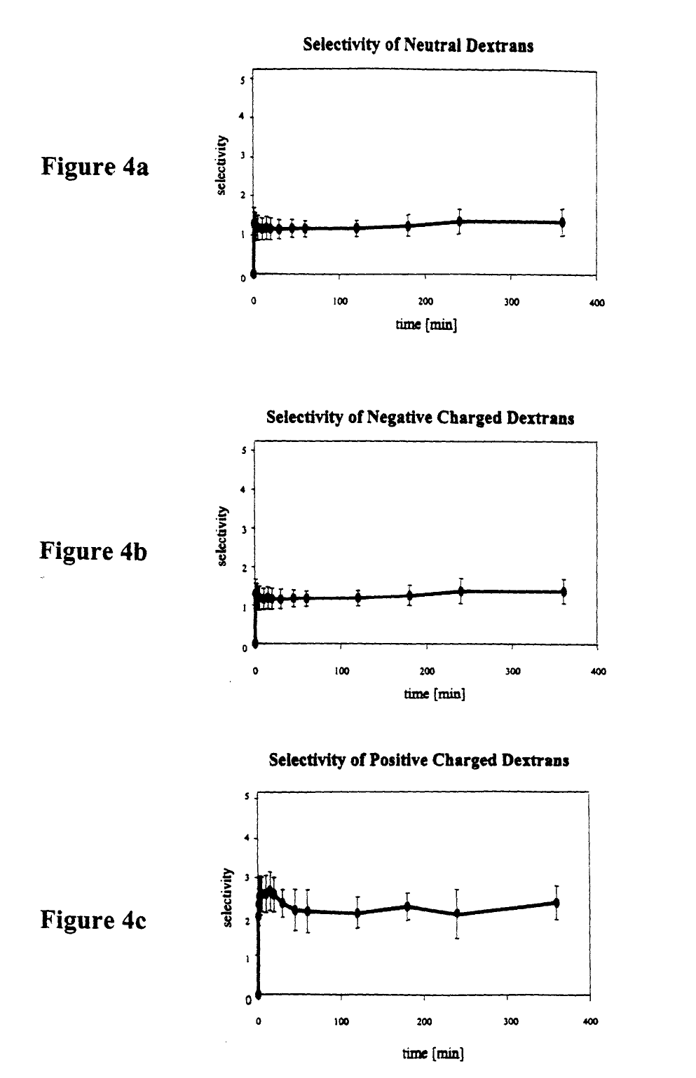

The present invention is further based on the finding that binding affinity of charged dextrans to one type of activated vascular site, specifically the angiogenic vascular endothelial cells located at a tumor site in vivo, increases with net positive charge of the molecule. As shown in FIGS. 4A-C, selectivity of dextran molecules with a net positive charge is greater than corresponding molecules that are neutral or have negative charge. For purposes of the present disclosure, the pI of negative dextrans is considered to be about 3; for neutral dextrans about 7 and for positive dextrans about 10. The results presented herein indicate that cationic macromolecules adhere to the negatively charged binding sites located on the cell surface or the glycocalyx of the endothelium, which reflects an increased local concentration of such agents at an activated vascular site as well as a local concentration gradient that favors the extravasation of such constructs through the relatively leaky endothelial cell walls and into the target tissue.

D. Vascular Permeability in a Human Tumor Xenograft: Molecular Charge Dependence

In normal tissues, the luminal endothelial membrane is negatively charged (Curry et al., 1987; Turner et al., 1983; Baldwin et al., 1991). Thus, it restricts the extravasation of anionic macromolecules, as has been demonstrated in vitro in cultured endothelial cell monolayer (Sahagun et al., 1990) and various normal tissues (Jain et al., 1997). As noted above, Adamson et al. (1988) have demonstrated that the microvascular permeability to (x-lactalbumin (MW=14,176; net charge −10) is approximately 50% of that to ribonuclease (MW=13,683; net charge +4), suggesting that the microvascular permeability for the positively charged molecules in normal tissues is higher than the permeability for the negative ones. The transport restriction of anionic macromolecules is crucial for maintaining a fluid homeostasis in the body, due to the osmotic effect (Curry, 1984).

However, tumor vessels are significantly different from normal vessels. The role of molecular charge in the transport across tumor vessel wall is still unknown. The present invention discloses the effect of molecular charge on transport processes across the tumor vessel barrier.

The present invention is based in part on the observation that positively charged molecules may accumulate at higher concentrations in angiogenic vessels of solid tumors compared to the similar sized compounds with neutral or negative charges. Following the higher accumulation, these positively charged molecules may extravasate faster from such tumor vessels to the tumor tissue. Table 4 (Example 5) shows that tumor vascular permeability of cationized BSA (pI-range: 8.6-9.1) and IgG (pI: 8.6-9.3) is more than two-fold higher (4.25 and 4.65×10⁻⁷ cm/s) than that to the anionized BSA (pI≈2.0; 1.11×10⁻⁷ cm/s) and IgG (pI 3.0-3.9; 1.93×10⁻⁷ cm/s). Accordingly, cationization which increases the pI or zeta potential of a molecule may be an effective approach for improving delivery of diagnostic or therapeutic agents, as well as gene therapy vectors, and other macromolecules to solid tumors.

E. Uptake of Neutral and Cationic Rhodamine-labeled Liposomes by Human Endothelial Cell Cultures (HUVEC)

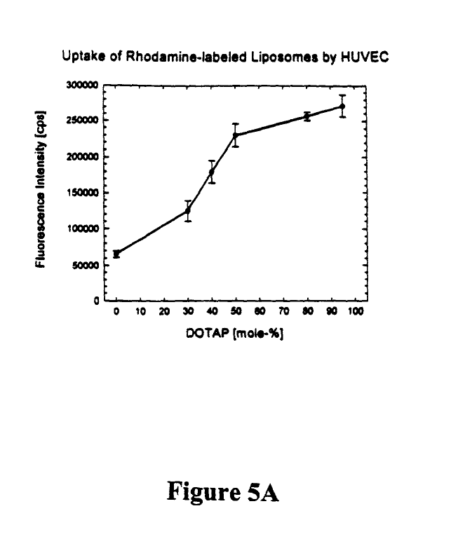

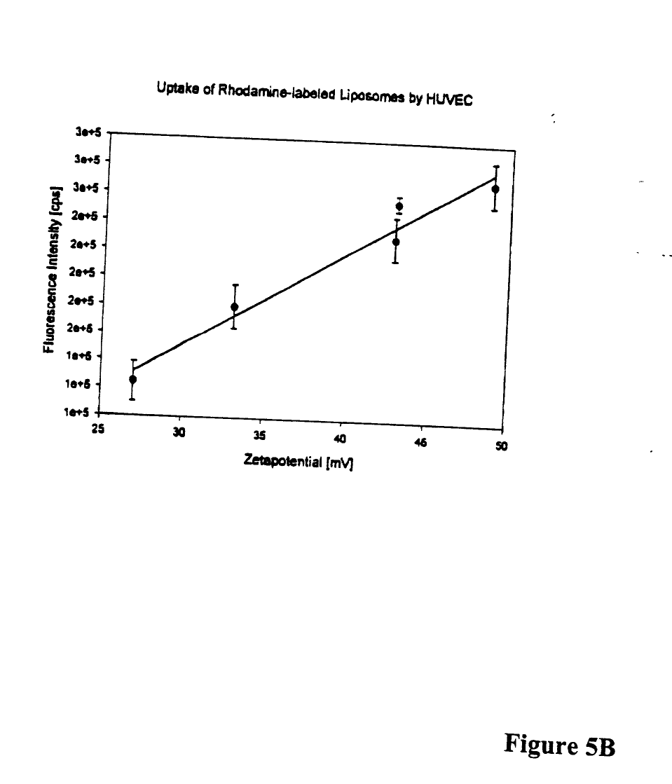

The present invention is further based on the finding that uptake of neutral and cationic rhodamine-labeled liposomes by HUVEC parallels the data discussed above correlating zeta potential and mol % of DOTAP. As shown in FIG. 5A, there is a relatively constant increase in fluorescence intensity from 0 to 50 mol % of DOTAP. At concentrations of greater than 50 mol % of DOTAP, fluorescence intensity levels off. Based on such data, the present inventors contemplate that liposomes intended for use in the methods and compositions of the present invention will preferably comprise about 20 to 60 mol % DOTAP or other cationic lipid for targeting endothelial cells at physiologic pH, and more preferably about 50 mol % as indicated above. FIG. 5B, which shows measurements of fluorescence intensity vs zeta potential, indicates that the relationship between fluorescence intensity measured in HUVEC cells and zeta potential is relatively linear, Thus, if zeta potential of the labeled liposome is not further increased, then the uptake of labeled liposomes by HUVEC would not increase.

F. Modification of Compounds to Increase their Charge (i.e. Isoelectric Point or Zeta Potential)

Various techniques are available to increase the isoelectric point or zeta potential of diagnostic, imaging and therapeutic agents by derivatizing or otherwise modifying such agents. For example the Morgan et al. U.S. Pat. Nos. 5,635,180 and 5,322,678 and Khawli et al. U.S. Pat. No. 5,990,286 describe various techniques for modifying protein compositions to modulate their net charge. Similarly, Rok et al. in Renal Failure 20 (2): 211-217 (1998) shows various therapeutic formulations in which a carrier molecule for an active ingredient has been modified.

Such techniques for the modification, derivatization and recombinant expression of products are generally applicable to antibodies, antibody fragments, growth factors, hormones, or other protein active agents. Other techniques will be appropriate for the modification and derivatization to increase the charge (isoelectric point) of various targeting and carrier moieties to which an active ingredient would be coupled. Such carriers include, for example, various polymers which include polyvinylpyrrolidone, pyran copolymer, polyhydroxy-propyl-methacrylamide-phenol, polyhydroxyethyl-aspartamide-phenol, or polyethylene oxide-polylysine substituted with palmitoyl residues. Useful cation forming agents include ethylene diamine via and EDCI reaction with a carboxyl group on the protein. A specific example is hexamethylenediamine. Other cationic agents include but are not limited to hexamethylenediamine, triethylene tetraamine, 4-dimethylamino butylamine, N,N-dimethylaminoethyl amine, and dimethylamino benzaldehyde.

Furthermore, active ingredients bearing a suitable net positive zeta potential according to the present invention may also be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as stents, tubing, and the like.

Persons skilled in the art of modifying compounds to modulate their net charge will be familiar with other relevant modification techniques. See, for example, U.S. Pat. No. 5,990,179 (1999) to Gyory et al. which describes composition and methods to increase the positive charge of drugs, albeit intended primarily to enhance their transdermal delivery, although the disclosed techniques are relevant to the compositions and methods of the present invention.

Examples of diagnostic, imaging, and therapeutic agents that would benefit from modifications increasing their charge include but are not limited to etherlipids, alkyllysolecithins, alkyllysophopholipids, lysolipids, alkylphospholipids. It is pointed out that these agents are cytostatic and that they can constitute a part of membrane bilayer of a liposome compositions. Specific examples of such agents include but not limited to as 1-O-octadecyl-2-O-methyl-rac-glycero-3-phosphocholine, 1-O-Hexadecyl-2-O-methyl-sn-glycerol, Hexadecyl phosphocholine, Octadecylphosphocholine.

G. Diagnostic and Imaging Labels

As one aspect of the present invention, positively charged molecules can be used as diagnostic markers for imaging tumors that have induced the growth of angiogenic endothelial cells. A preferred application of the present invention for imaging purposes involves the use of magnetic resonance as a diagnostic tool. For agents appropriate for administration in a liposomal form, the skilled artisan will be aware of various protocols for the preparation of liposomes that can be formulated with the ranges of cationic and non-cationic components to produce liposomes having a preferred zeta potential as described above (Szoka et al, 1980). Magnetosomes targeting endothelial cells can also be obtained using the same cationic and non-cationic compounds that are used for liposomal formulations described above. For large molecules, particularly proteins, other appropriate techniques, such as those provided above, may be used to prepare agents having isoelectric points in the preferred ranges. Carriers, such as biopolymers, microemulsions, iron oxide particles, could be used for preparing agents having the preferred isoelectric points. Alternatively, the agent can be modified by cationization.

Persons skilled in the art also will appreciate that magnetic resonance imaging (MRI) is currently one of the most sensitive, non-invasive way of imaging soft tissues of the body. Unlike a CT scan or conventional X-ray, this type of scanning device does not use radiation; instead, it makes use of magnetic fields that interact with the hydrogen atoms found in the water contained in all body tissues and fluids. During the course of an MRI scan, computers translate the increased energy of various hydrogen nuclei into cross-sectional images of the tissue to be studied. The scanning procedure is very sensitive, and can often detect tumors that would be missed on a CT scan. Many different types of tissue and tumors can be imaged by MRI, including, but not limited to, brain, mammary, and any solid tumor found in any soft tissue in the body (including liver, pancreas, ovaries, etc.).

To increase the sensitivity of MRI (as well as CT) scans, various contrast media are used. Although “macromolecular MRI contrast media” (MMCM) have been known for some time, these media only recently have found diagnostic uses (Kuwatsuru et al., 1993). Several classes of compounds have potential as contrast agents in MRI. These classes include superparamagnetic iron oxide particles, nitroxides, and paramagnetic metal chelates (Mann et al., 1995). A strong paramagnetic metal generally is preferred. Normally, paramagnetic lanthanides and transition metal ions are toxic in vivo. Thus, it is necessary to incorporate these compounds into chelates with organic ligands. By enhancing the targeting of such chelated metals to the vicinity of angiogenic endothelial cells according to the present invention, it is possible to reduce the total dose of imaging composition otherwise required.

Acceptable chelates are known in the field. They include: 1,4,7,10-tetraazacyclododecane-N,N′,N″,N′″-tetraacetic acid (DOTA); 1,4,7,10-tetraazacyclododecane-N,N′,N″-triacetic acid (DO3A); 1,4,7-tris(carboxymethyl)-10-(2-hydroxypropyl)-1,4,7,10-tetraazacyclododecane (HP-DO3A); diethylenetriaminepentaacetic acid (DTPA); DTPA coupled to polymers (e.g., to poly-L-lysine or polyethyleneimine); and many others. Paramagnetic metals of a wide range are suitable for chelation. Suitable metals are those having atomic numbers of 22-29 (inclusive), 42, 44 and 58-70 (inclusive), and having oxidation states of 2 or 3. Those having atomic numbers of 22-29 (inclusive), and 58-70 (inclusive) are preferred, and those having atomic numbers of 24-29 (inclusive) and 64-68 (inclusive) are more preferred. Examples of such metals are chromium (III), manganese (II), iron (II), cobalt (II), nickel (II), copper (II), praseodymium (III), neodymium (III), samarium (III), gadolinium (III), terbium (III), dysprosium (III), holmium (III), erbium (III) and ytterbium (III). Chromium (III), manganese (II), iron (III) and gadolinium (III) are particularly preferred, with gadolinium (III) being the most preferred. See, e.g., published PCT application WO 94/27498 for additional information about such paramagnetic agents.

Typically, contrast media for the imaging of tumors is administered by the parenteral route, e.g., intravenously, intraperitoneally, subcutaneously, intradermally, or intramuscularly. Thus, the contrast media is administered as a composition that comprises a solution of contrast media dissolved or suspended in an acceptable carrier, generally an aqueous carrier. The concentrations of MMCM varies depending on the strength of the contrast agent but typically ranges from about 0.1 μmol/kg to about 100 μmol/kg. A variety of aqueous carriers are known, e.g., water, buffered water, 0.9% saline, 5% glucose, 0.3% glycine, hyaluronic acid and the like. These compositions may be sterilized by conventional, well known sterilization techniques, or may be sterile filtered.

Brasch et al. (U.S. Pat. No. 6,009,342) teaches the use of contrast agents attached to a large backbone for macromolecular contrast media imaging (MCMI), a quantitative method for estimating the microvascular permeability of tumors, more particularly breast tumors. The backbone can be a protein, such as albumin, a polypeptide, such as poly-L-lysine, a polysaccharide, a dendrimer, or a rigid hydrocarbon or other compound with a small molecular weight but a larger effective molecular size. The preferred backbones are compounds that, when passed through a gel filtration matrix, behave similarly to a peptide of 30 kDa.

Other methods for imaging tumors include CT scans, positron emission tomography (PET), and radionuclide imaging. The contrast media for CT scans includes all molecules that attenuate x-rays. As would be known to persons skilled in the imaging field, for positron emission tomography and radionuclide imaging, short lived radioisotopes are preferred. Similarly, it would be known that all positron emitting isotopes are useful as contrast media for positron emission tomography, and all γ-ray emitting isotopes are useful for radionuclide imaging.

Ultrasonic imaging is another method of imaging the body for diagnostic purposes. There are two general types of ultrasound contrast agents; positive contrast agents and negative contrast agents. Positive contrast agents reflect the ultrasonic energy and thus they produce a positive (light) image. Correspondingly, negative contrast agents enhance transmissibility or sonolucency and thus produce a negative (dark) image. A variety of substances—gases, liquids, solids, and combinations of these—has been investigated as potential contrast-enhancing agents. Examples of solid particle contrast agents disclosed in U.S. Pat. No. 5,558,854 include but not limited to IDE particles and SHU454. European Patent Application 0231091 discloses emulsions of oil in water containing highly fluorinated organic compounds for providing enhanced contrast in an ultrasound image. Emulsions containing perfluorooctyl bromide (PFOB) have also been examined as ultrasound imaging agents. U.S. Patent No. 4,900,540 describes the use of phospholipid-based liposomes containing a gas or gas precursor as a contrast-enhancing agent.

Additionally, labeled monoclonal antibodies have been used to localize diseased or damaged tissue. Useful labels include radiolabels (i.e., radioisotopes), fluorescent labels and biotin labels. Among the radioisotopes that can be used to label antibodies or antibody fragments that are suitable for localization studies are gamma-emitters, positron-emitters, X-ray-emitters and fluorescence-emitters. Appropriate radioisotopes for labeling antibodies include Iodine-131, Iodine-123, Iodine-125, Iodine-126, Iodine-133, Bromine-77, Indium-111, Indium-113m, Gallium-67, Gallium-68, Ruthenium-95, Ruthenium-97, Ruthenium-103, Ruthenium-105, Mercury-107, Mercury-203, Rhenium-99m, Rhenium-105, Rhenium-101, Tellurium-121m, Tellurium-122m, Tellurium-125m, Thulium-165, Thulium-167, Thulium-168, Technetium-99m and Fluorine-18. The halogens can be used more or less interchangeably as labels since halogen-labeled antibodies and/or normal immunoglobulins would have substantially the same kinetics and distribution and a similar metabolism. The gamma-emitters, Indium-11 and Technetium-99m, are preferred because such radiometals are detectable with a gamma camera and have favorable half lives for imaging in vivo. Antibody can be labeled with Indium-111 or Technetium-99m via a conjugated metal chelator, such as DTPA (diethlenetriaminepentaacetic acid). See, e.g., Krejcarek et al. (1977); Khaw et al (1980); U.S. Pat. Nos. 4,472,509; and 4,479,930). Fluorescent compounds that are suitable for conjugation to a monoclonal antibody include fluorescein sodium, fluorescein isothiocyanate, and Texas Red sulfonyl chloride (DeBelder et al., 1975).

The present invention also contemplates non-fluorescent dye, for example patent blue V. Hirnle et al. (1988) describe encapsulating patent blue V in liposomes.

H. Therapeutic Formulations and Delivery Systems

Formulations of the present invention include, but not limited to, therapeutic, diagnostic, and imaging compositions. Contemplated compositions can include an active ingredient such as a cytostatic or cytotoxic agent. Examples of cytostatic or cytotoxic agents include, but not limited to, taxanes, inorganic complexes, mitose inhibitors, hormones, anthracyclines, antibodies, topoisomerase inhibitors, antiinflammtory agents, angiogenesis inhibitors, alkaloids, interleukins, cytokines, growth factors, proteins, peptides, tetracyclines, and nucleoside analogs. Specific examples of taxanes include paclitaxel and docetaxel. Specific examples of inorganic complexes include cisplatin. Specific examples of anthracyclines include daunorubicin, doxorubicin, and epirubicin. Specific examples of inhibitors of angiogenesis include angiostatin. Specific examples of alkaloids include vinblastin, vincristin, navelbine, and vinorelbine. Specific examples of nucleoside analogs include 5-fluorouracil and others. Also contemplated active agents are therapeutically effective fragments of cytokines, interleukins, growth factors, proteins, and antibodies.

Various delivery systems are known and can be used for the administration of therapeutic compositions that include a positively charged diagnostic, imaging or therapeutic agent or a positively charged carrier for such agents. For example, encapsulation in liposomes, microparticles and microcapsules as well as magnetosomes have been described for numerous diagnostic, imaging and therapeutic products. In some instances, these formulations result in receptor-mediated endocytosis (Wu and Wu, 1987, J. Biol. Chem. 262:4429-4432). In general, appropriate methods for the administration of such compositions to a subject include but are not limited to intradermal, intramuscular, intraperitoneal, intravenous, intraarterial, subcutaneous, intranasal, epidural, and oral routes. Alternative systemic administration include transmucosal and transdermal administration using penetrants such as bile salts or fusidic acids or other detergents. Moreover, therapeutic compositions can be administered to a tumor site by direct intratumoral injection.

Compositions according to the present invention may be administered by any convenient route, for example by infusion or bolus injection, by absorption through epithelial or mucocutaneous linings (e.g., oral mucosa, rectal and intestinal mucosa, etc.) and may be administered in combination with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, optionally with an aerosolizing agent.

It may be desirable to administer the pharmaceutical compositions of the present invention locally to the area in need of treatment. This may be achieved, for example and not by way of limitation, by topical application, by injection, by means of a catheter, by means of a suppository, or by means of an implant, said implant being of a porous, non-porous, or gelatinous material, including membranes, such as sialastic membranes, or fibers.

The compositions of the present invention also can also be delivered in a controlled release system. For example, a pump may be used (see Langer, supra; Sefton, CRC Crit. Ref. Biomed Eng. 14:201 (1987); Buchwald et al., Surgery 88:507 (1980); Saudek et al., N. Engl. J. Med. 321:574 (1989)). In another embodiment of the present invention, polymeric materials can be used (Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); Levy et al., Science 228:190 (1985); During et al., Ann. Neurol. 25:351 (1989); Howard et al., J. Neurosurg. 71:105 (1989)). Additionally, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

The present invention also contemplates a wide variety of pharnaceutical compositions and formulations consistent with the research findings presented herein. Such compositions comprise a therapeutically effective amount of a therapeutic agent, and a pharmaceutically acceptable carrier. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol —solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, -glycerol, propylene, glycol, water, ethanol and the like. The composition, if desired, can also contain minor amounts of wetting or emulsifying agents, or pH buffering agents. Such compositions can take the form of solutions, suspensions, emulsion, tablets, pills, capsules, powders, sustained-release formulations and the like. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic composition, preferably in purified form, together with a suitable amount of carrier so as to provide the form for proper administration to the patient.

The overall formulation should suit the mode of administration. Thus, the compositions according to the present invention are formulated in accordance with routine procedures adapted, for example, to the intravenous administration to human beings. Typically, compositions for intravenous administration are solutions in sterile isotonic aqueous buffer. Where appropriate, the compositions may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampule indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients may be mixed prior to administration.

The amount of the diagnostic, imaging and therapeutic compositions ofthe present invention which will be effective in the diagnosis, monitoring, imaging and treatment of a particular disorder or condition will depend on the nature of the disorder or condition, and can be determined by standard clinical techniques. In addition, in vivo and/or in vitro assays may optionally be employed to help identify optimal dosage ranges. The precise dose to be employed in any particular formulation will also depend on the route of administration, and the seriousness of the disease or disorder, and should be decided according to the judgment of the practitioner and each patient's circumstances. In general, however, where known compounds are modified to increase their net positive zeta potential according to the methods described herein, the dosage of active ingredient may be lower than the dose of the unmodified compound.

I. Administration of Compositions for the Imaging and Treatment of Tumors

The active ingredients of the present invention can be administered via routes of administration deemed to be appropriate by the attending oncologist or other physician. Such route also would include direct injection into a tumor mass or in any manner that provides for delivery of the compositions of the present invention into the vicinity of angiogenic endothelial cells. The dosage administered will be dependent upon the age, health, and weight of the recipient, kind of concurrent treatment, if any, frequency of treatment, and the nature of the effect desired as is well known to oncologists.

In practicing the methods of this invention, the compounds of this invention may be used alone or in combination, or in combination with other diagnostic, imaging and therapeutic agents. In certain preferred embodiments, the compounds of this invention may be coadministered along with other compounds typically prescribed for these conditions according to generally accepted medical practice, including anti-angiogenic agents, such as angiostatin or endostatin expression vectors or proteins, or other anti-cancer therapeutics. The compounds of this invention can be utilized in vivo, ordinarily in mammals, such as humans, sheep, horses, cattle, pigs, dogs, cats, rats and mice, or in vitro.

Therapeutically effective dosages may be determined by either in vitro or in vivo methods. For each particular compound of the present invention, individual determinations may be made to determine the optimal dosage required. The range of therapeutically effective dosages will be influenced by the route of administration, the therapeutic objectives and the condition of the patient, as well, for example, by the nature, stage and size of a tumor. For injection by hypodermic needle, it may be assumed the dosage is delivered into the body's fluids. For other routes of administration, the absorption efficiency must be individually determined for each compound by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be readily determined by one skilled in the art. Typically, applications of compound are commenced at lower dosage levels, with dosage levels being increased until the desired effect is achieved.

While individual needs vary, determination of optimal ranges of effective amounts of each component is within the skill of the art. Generally, the optimal dosage will be equal to or less than the corresponding dose for therapeutic agents that have not been modified or derivatized in some way as to increase their net zeta potential. It is contemplated that therapeutic agents modified to exhibit an increased net zeta potential for selective targeting may have a higher safety level and lower toxicity level and may be administered at higher doses. More generally, the compounds of the invention can be administered intravenously or parenterally in an effective amount within the dosage range of about 0.01 mg to about 50 milligram/kg, preferably about 0.05 mg to about 5 mg/kg and more preferably about 0.2 mg to about 1.5 mg/kg on a regimen in a single or 2 to 4 divided daily doses and/or continuous infusion.

J. Treatment and Imaging of Various Diseases Exhibiting Activated Vascular Sites

There are several neoplastic and non-neoplastic diseases associated with proliferating or angiogenic epithelial cells as found in certain types of activated vascular sites. As discussed in Davis-Smyth et al., U.S. Pat. No. 5,952,199, these diseases include solid and metastatic tumors, and diseases such as rheumatoid arthritis, psoriasis, atherosclerosis, diabetic retinopathy, retrolenta fibroplasia, neovascular glaucoma, age-related macular degeneration, hemangiomas, immune rejection of transplanted corneal or other tissue, and chronic inflammation.

Conventional therapies for these diseases are varied. For example, cancers may be treated by a wide variety of chemotherapeutics. Rheumatoid arthritis is often treated with aspirin or aspirin substitutes such as ibuprofen, corticosteroids or immunosuppressive therapy. Merck Manual (1992) 16th ed., pp. 1305-12. Atherosclerosis treatment is directed towards symptomatic conditions or risk factors, such as reducing circulating cholesterol levels or angioplasty. Merck Manual (1992) 16th ed., pp. 409-412. Diabetes mellitus can induce a range of condition, including diabetic atherosclerosis and diabetic retinopathy, which can be treated by controlling the primary diabetes or associated conditions such as blood pressure. Merck Manual (1992) 16th ed., pp. 412-413, 1106-1125, 2383-2385. Psoriasis is most commonly treated with topical ointments and steroid treatments. Merck Manual (1992) 16th ed., pp. 2435-2437. Retrolenta fibroplasia is best treated by preventative oxygen and vitamin E treatments, although cryotherapeutic ablation may also be required. Merck Manual (1992) 16th ed., pp. 1975-1976. From the foregoing, it is clear that these angiogenesis-associated diseases do not share common treatment indications despite their shared angiogenic association.

Other diseases associated with activated vascular sites include inflammatory diseases, such as nephritis. Recently, Iruela-Arispe et al. (1995) described the participation of glomerular endothelial cells in the capillary repair induced in response to glomerulonephritis. In many glomerular diseases, severe injury to the mesangium may occur, leading to matrix dissolution and damage to glomerular capillaries. Although the destruction of the glomerular architecture may lead to permanent injury, in some cases spontaneous recovery occurs. Iruela-Arispe et al. showed proliferation of endothelial cells from days 2 to 14 after severe injury to the mesangium, in association with repair of the glomerular capillaries. The initial endothelial cell proliferation is associated with basic fibroblast growth factor and the later glomerular endothelial cell proliferation is associated with an increase of vascular permeability factor/endothelial cell growth factor and an increase of flk, a VPF/VEGF receptor. This indicates that glomerular endothelial cells play an active role in the glomerular response to injury, and that the therapeutic, imaging and diagnostic compositions of the present invention would be useful in connection with inflammatory conditions such as glomerulonephritis.

In addition, the inhibition or prevention of angiogenesis provides a relatively new and more global mechanism of treating a variety of angiogenesis-associated diseases. Thus, an aspect of the present invention is the targeting of agents that will selectively accumulate at activated vascular sites, such as in the vicinity of angiogenic or proliferating endothelial cell, to cause the death of such angiogenic cells or the cells of tumors that have induced the angiogenesis of such cells and neovasculature. In this regard, highly toxic agents have been described in copending U.S. Provisional Patent Application Serial No. 60/163,250 that appropriately may be formulated, for example, in liposomes having a preferred zeta potential according to the present specification.

K. Combination or Co-administration Therapies

As contemplated, the present invention also relates to the combination or co-administration of the compounds disclosed herein by the associated inventive methods, together with the administration of other therapies, angiogenesis inhibitors and/or other anti-tumor agents. Such other therapies, angiogenesis inhibitors and agents are well known, for example, to ophthalmologists and oncologists. Such other agents and associated methods to be used in combination with the constructs and methods of the present invention include conventional chemotherapeutic agents, radiation therapy, immunomodulatory agents, gene therapy, and the use of various other compositions such as immunotoxins and anti-angiogenic formulations, such as angiostatin or endostatin, as are disclosed, for example, in U.S. Pat. No. 5,874,081 to Parish et al. (1999) and U.S. Pat. No. 5,863,538 to Thorpe et al. (1999) or are otherwise known in the art. Combination or co-administration therapies based on the present invention and the conventional therapies for angiogenesis associated diseases, such as discussed above, are also particularly contemplated.

L. Wound Healing

It also is expressly contemplated that compositions and formulations according to the present invention may be selectively targeted to enhance the treatment of wounds or other such activated vascular sites in order to enhance the healing process, as opposed to treatment of pathological conditions associated with activated vascular sites.

Growth factors, such as fibroblast growth factor and vascular endothelial cell growth factor, promote cell proliferation and differentiation during the normal wound healing process. The fibroblast growth factor family includes at least seven polypeptides that have been shown to stimulate proliferation in various cell lines including endothelial cells, fibroblasts, smooth muscle cells and epidermal cells. Members of the family include acidic fibroblast growth factor (FGF-1), basic fibroblast growth factor (FGF-2), int-2 (FGF-3), Kaposi sarcoma growth factor (FGF-4), hst-1 (FGF-5), hst-2 (FGF-6) and keratinocyte growth factor; (FGF-7) (Baird and Klagsbrun, Ann. N.Y. Acad. Sci. 638: xiv, 1991). Vascular endothelial growth factor (VEGF) also known as vascular permeability factor (VPF) is a highly selective mitogen for vascular endothelial cells (Ferrara et al., 1992). VPF has been found to be responsible for persistent microvascular hyperpermeability to plasma proteins even after the cessation of injury, which is a characteristic feature of normal wound healing. This suggests that VPF plays an important role in wound healing (Brown et al., 1992).

For wound healing, growth factors can be encapsulated in liposomes and delivered to the target site by local injection of the liposomal composition. Liposomes are available commercially from a variety of suppliers. Alternatively, liposomes can be prepared according to methods known to those skilled in the art, for example, as described in U.S. Pat. No. 4,522,811. U.S. Pat. No. 5,879,713 teaches preparation of liposome formulations by dissolving appropriate lipid(s) (such as stearoyl phosphatidyl ethanolamine, stearoyl phosphatidyl choline, arachadoyl phosphatidyl choline, and cholesterol) in an organic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension. Generally, the biologically active molecules, such as FGF or VEGF, are mixed with the liposome in a concentration which will release an effective amount at the targeted site in a patient.

M. Other Conditions Associated with Impaired Angiogenesis

It also is expressly contemplated that compositions and formulations according to the present invention may be selectively targeted to treat other conditions associated with impaired angiogenesis.

It has recently been shown that angiogenesis is impaired in aging. Reed et al. (2000) reported that delayed neovascularization is due in part, to slowed endothelial cell migration as a consequence of decreased collagenase activity. Accordingly, perturbations to enhance collagenase activity may increase microvascular endothelial cells migratory ability and angiogenic potential. Rivard et al. (1999) disclosed that impaired angiogenesis in old animals was the result of impaired endothelial finction including lower basal NO release, decreased vasodilation in response to acetylcholine, and a lower expression of VEGF in ischemic tissues. Thus, Rivard et al. (1999) concluded that angiogenesis responsible for collateral development in limb ischemia is impaired with aging as a consequence of age-related endothelial dysfunction and reduced VEGF expression. However, it seems that advanced age does not augment collateral vessel development and could be affected by exogenous angiogenic cytokines.

Yamanaka et al. (1999) reported that regeneration of impaired glomerular capillary networks plays an important role in the repair process of glomerular lesions. Glomerular endothelial cell injury influences the progression and repair process of glomerular diseases. When a glomerular lesion is severe, angiogenesis is prevented due to endothelial cell injury, with subsequent sclerosis taking place in the impaired region. Inevitably, glomerular endothelial cell injuries affect mesangial and epithelial cells. It is therefore contemplated that the progression of renal disease could be modulated by promoting angiogenesis of endothelial cells.

Raynaud's phenomenon is characterized by sensitivity of the hands to cold due to spasms of the digital arteris, resulting in blanching and numbness of the fingers. It is a circulatory disorder caused by insufficient blood supply to the hands and feet. Studies have suggested that changes in the nervous system at either the peripheral level or the central level are linked to the dysfunction of endothelial cells. Cerinic et aL(1997) proposed the use of therapeutic angiogenesis (regeneration of vessels) for the treatment of Raynaud's disease and the loss of angiogenesis in diffuse scleroderma.

N. Treatment of the Brain

It also is expressly contemplated that compositions and formulations according to the present invention may be selectively targeted to cross the blood brain barrier by presenting an appropriate net positive charge to the endothelial cells at the blood brain barrier.

In light of the foregoing general discussion, the specific examples presented below are illustrative only and are not intended to limit the scope of the invention. Other generic and specific configurations will be apparent to those persons skilled in the art.

### EXAMPLES

### Example 1

### Synthesis of Charged Dextran Coated Iron Oxide Particles

Dextran-stabilized iron oxide particles were prepared as described in the literature (Papisov et al., 1993). Oxidation of the particles (for example with periodate) produced aldehyde groups on the surface of each colloid. Subsequently, the aldehyde groups can be reacted with the amine function of various reagents, yielding products with different net charge. For example, coupling with phosphatidylethanolamine (net charge zero) or with another neutral molecule having a free amine function yields a product with a negative charge. Coupling with a dendrimer, with polylysine, with a protein with positive net charge or with another suitable molecule with excess positive charge in the appropriate molar ratio yields a product with a net positive charge. The unmodified and unoxidized dextran-stabilized iron oxide particles were used as representative of uncharged molecules.

1. Preparation of Dextran-Stabilized Iron Oxide Particles (Neutral Charge).

The classical route of co-precipitation of magnetite in the presence of a coating material such as dextran was applied. The reaction proceeds in two steps. First, FeCl₂ and FeCl₃ are mixed with dextran and precipitated in alkaline medium, yielding Fe(OH)₂ and Fe₂O₃.nH₂O. Upon heating, water is eliminated and supraparamagnetic crystals of Fe₃O₄ are obtained. Redispersing the particles in water yielded particles with various size distributions. Particles with a Z_{average} (mean particle size of an ensemble of particles fitted to a monomodal distribution) of 80 to 120 mn (Zetasizer 3000, Malvern Instruments) were utilized. Typically, one ml solution contains approximately 0.2 mmol dextran and 31 mg which is 0.55 mmol Fe. The zeta potential of these particles is about 0 to −15 mV at pH 7 in 0.05 M KCl.

2. Oxidation of Iron Oxide Particles.

Oxidation of dextran coated iron oxide particles was obtained by modifying the procedure of Bogdanov et al. (Bogdanov Jr. et al., 1994). Dextran-coated iron oxide particles were mixed with sodium periodate in an approximate molar ratio of 6 mol dextrose to 1 mol IO₄-in aqueous solution (30 min, pH 6). The reaction was stopped by adding ethyleneglycol (about 300 fold or higher molar excess). Subsequently, the solution was dialyzed against 0.15 M NaCl.

3. Preparation of Positively Charged Particles.

The oxidized iron oxide particles obtained above were used to prepare positively charged particles. An aqueous solution of polylysine (2 μmol) was mixed with about 20 μmol of sodium borate (pH 9) and modified iron oxide particles from step 2 containing 300 μmol iron and 100 μmol dextrose. The emulsion was dialyzed against 0.15 M NaCl and used for cell culture experiments. The zeta potential of these particles was about +50 mV (H 7.5).

4. Negatively Charged Particles.

Negatively charged iron oxide particles, which are coated with a double layer of lauric acid are commercially available from Berlin Heart AG. The zeta potential of these particles is about −30 to −40 mV (at about pH 7.0).

### Example 2

### Alternative Synthesis of Charged Dextran Particles.

Neutral, polycationic and polyanionic dextran can be purchased from Amersham Pharmacia Biotech. While the neutral dextran can be obtained in a wide variation of size classes (from 10,000 to 2,000,000 Daltons), the charged dextrans are available only in one size (500,000 Daltons). The cationic dextran is a diethylaminoethyl ether (DEAE) derivative, the anionic dextran is a sulfate. As described below, DEAE dextran and dextran sulfate having other molecular sizes were synthesized by modifying well known procedures.

1. Preparation of Dextran Particles with a Defined Size.

Before derivatization, dextran molecules were separated into different classes based on their size similar to a method described by Isaacs et al. (1983). Thus, subsequent characterization (e.g., charge density, isoelectric focusing) can be used to determine the ratio of charged functional groups per molecule dextran.

Dextran was purchased from Pharmacia, Upsala, Sweden in three different size classes (10,000, 70,000 and 500,000 Daltons) that were used as starting material in each case. Next, the molecular size of the dextran was checked by gel chromatography. To do this, the dextran with 10,000 Daltons was loaded onto a Sephacryl S-200 column (Pharmacia, 75 cm length, 5 cm inner diameter) and eluted with 0.05 M ammonium bicarbonate (pH 8.2). Then, 20 ml fractions were collected and analyzed for hexose content as described by Mokrasch et al. (1954). The 70,000 Dalton dextran was analogously chromatographed on a AcA 22 column (LKB, Bromma, Sweden, 96×2.5 cm). Next, 9 ml fractions were collected. The 500,000 Dalton dextran was chromatographed on a Sepharose 6B column (Pharmacia, 96×2.5 cm) and 9 ml fractions were collected and analyzed. For each dextran type, the fractions with maximum dextran content were collected and pooled and amounted to approximately 70% of the starting material. From each dextran pool, the material was lyophilized for further use.

The isoelectric point of the dextran fractions was determined by isoelectric focussing on Pharmalyte gel (Amersham Pharmacia Biotech) covering pH 3 to 11 and was found to be at pH 7.

2. Preparation of Polyanionic Dextran.

Dextran sulfate was prepared as described by Nagasawa et al. (1974). Thus, 162 mg dextran (1 mmol glucose), 225 mg 8-quinolyl sulfate (1 mmol) and 67 mg CuCl₂ (0.5 mmol) were mixed in 10 ml anhydrous dimethylformamide and stirred at 40° C. for 5 hours. Subsequently, the reaction mixture was diluted with 50 ml water which led to formation of a precipitate. The precipitate was removed by filtration and the filtrate was passed through a column of Dowex 50W (X8, H⁺, 20-50 mesh). The effluent and washings were combined, neutralized with 2 N NaOH and dialyzed overnight against water. Next, the dialyzed solution was concentrated to 2.5 ml in vacuo, and was added dropwise into 45 ml of ethanol which led to precipitation of sodium dextran sulfate. This precipitate was separated by centrifugation, washed with ethanol and dried over P₂O₅ in vacuo for 3 hours at 80° C.

The isoelectric point (IEP) of each complex was determined with isoelectric focusing on Pharmalyte gel (Amersham Pharmacia Biotech) covering pH 2.5 to 5. The dextran sulfate from the 10,000 Dalton fraction had its IEP at pH 3, the 70,000 Dalton fraction below pH 2.5 and the 500,000 Dalton fraction at 2.8.

In each product, sulfate was determined gravimetrically as BaSO₄ (Harris et al., 1997) while the dextran was quantified with anthrone (Mokrasch et al, 1954). The molar ratio of dextran/sulfate was approximately 46 for the 10,000 Dalton fraction, 190 for the 70,000 Dalton fraction and 54 for the 500,000 Dalton fraction.

3. Preparation of Polycationic Dextran.

The DEAE dextran was prepared as described by McKeman et al. (1960). Thus, 6 g of dextran was dissolved in NaOH solution (4 g NaOH in 17 ml water) and cooled to 0° C. 2-chlorotriethylamine hydrochloride (3.5 g in 4.5 ml water) was added under stirring and the temperature was increased to 80° C. for 35 min. After cooling, ethanol was added while stirring, which led to precipitation of DEAE dextran. The product was separated by centrifugation, dissolved in water and precipitated again. This procedure was repeated until the supernatant was colorless. An aqueous solution of the product was finally neutralized with HCl, dialyzed and concentrated under vacuum. The DEAE dextran was isolated by lyophilization.

The isoelectric point (IEP) of each complex was determined with isoelectric focusing on Pharmalyte gel (Amersham Pharmacia Biotech) covering pH 8 to 10.5. The DEAE dextran from the 10,000 and from the 70,000 Dalton fractions had their IEPs above pH 10.5, and the 500,000 Dalton fraction had its IEP at pH 9.6.

In each product, nitrogen was determined by combustion analysis followed by GC analysis while the dextran was quantified with anthrone (Mokrasch et al, 1954). The molar ratio of dextran/amine was approximately 2 for the 10,000 Dalton fraction, 45 for the 70,000 Dalton fraction and 590 for the 500,000 Dalton fraction.

### Example 3

### Uptake of Charged Dextran Coated Iron Oxide Particles by HUVEC

Human endothelial cell cultures (HUVEC) were seeded at cell densities of 2×10⁴ cells per cm² in gelatin coated 10 cm² culture plates and cultured for 48 h in endothelial growth medium with 2% fetal calf serum at 37° C. and 5% CO₂ in a humidified atmosphere. The culture medium was removed, cells were washed with PBS and 1 ml of serum free endothelial basal medium was added. Charged dextran coated iron oxide particles were added to the cultures at a concentration of 50 μg Fe³⁺/ml. After 4 h of cultivation the medium was removed and the cultures were washed with 1 ml PBS. The removed culture medium and the washing solution were pooled and the iron concentration was measured by the thiocyanate reaction method described in Jander (1995). The cells were lysed with 500 μl concentrated HCl and the culture plates were washed with 500 μl PBS. The cell lysate and the washing solution were pooled and the iron concentration was measured by the thiocyanate reaction method described in Jander (1995).

The results of the foregoing experiment are presented in Table 1, below.

TABLE 1
Iron Concentration in Cell Lysates and Culture Media of HUVEC Cultures
After Incubation with Charged Dextran Coated Iron Oxide Particles.

| | | Cell lysate | Medium | Total | Recovery | Cell lysate | Medium |
|---|---|---|---|---|---|---|---|
| Particle | Charge | Fe³⁺ [μg] | Fe³⁺ [μg] | Fe³⁺ [μg] | [%] | % of total | % of total |
| Fe Coated with Lauric | negative | 11.04 | 27.35 | 38.39 | 78.48 | 28.7 | 71.3 |
| Acid | | | | | | | |
| FeDex-pLys | positive | 16.80 | 15.59 | 32.39 | 61.09 | 51.8 | 48.2 |
| FeDex | neutral | 5.38 | 23.84 | 29.22 | 70.92 | 18.4 | 81.6 |

The measured iron concentration in the cell lysates and culture media clearly show, that HUVEC uptake of positively charged dextran coated iron oxide particles is remarkably higher than uptake of neutral or negatively charged particles.

### Example 4

### Measurement of Zeta Potential of Liposomes

1. Measurement Principles

The zeta potential was measured with a Zetasizer 3000 (Malvern Instruments). In this experiment, the electrophoretic mobility depends on the charge density of the colloidal particle and is measured with Laser Doppler micro-electrophoresis. The particles are detected based on their light scattering behavior.

Measurements were carried out at 25° C. in several replicates. Between samples, a standard solution (latex particles with defined zeta potential) were repeatedly measured to ensure that the system is working correctly. Liposomes with varying content of a cationic component (DOTAP) were prepared (10 mM total lipid content).

2. Synthesis of Liposomes.

First, the film method is followed. Lipids are dissolved in chloroform in a round bottom flask, the flask is then rotated under vacuum until the lipids form a thin film. The lipid film is dried at 40° C. under a vacuum of 3 to 5 mbar for approximately 60 minutes. Subsequently, the lipids are dispersed in the appropriate volume of 5% glucose yielding a suspension of multilamellar lipid vesicles (10 mM lipid concentration). One day later, the vesicles are extruded (filtration under pressure) through membranes of appropriate size, typically between 100 and 400 mn (for zeta potential, all liposomes were extruded through 100 nm membranes).

For zeta potential measurements, formulations were diluted (1:25) in two different solvent systems: a) Tris-HCl buffer (pH 6.8 or 8.0, respectively) and b) 0.05 M solution of KCl, pH 7.0 (increased to 7.5 to 7.7 after sample was added)

The results of this experiment are described in Tables 2 and 3 below.

TABLE 2
Measurement of Seven Liposome Formulations (varying content
of DOTAP, neutral lipid DOPC (dioleoly-phosphatidylcholine)
or DOPE (dioleoyl-phosphatidylethanolamine) in Tris-HCl Buffer.
Sample conductivity approximately 3 mS/cm² (pH 6.8)
and 1 mS/cm² (pH 8), respectively.

| | DOTAP | Zeta | Zeta | |
|---|---|---|---|---|
| Liposome | content | potential in | potential in | Z_{ave in} |
| formulation | [mol %] | mV (pH 6.8) | mV (pH 8) | nm |
| DOTAP/DOPC = | 4 | +10.8 | +7.0 | 148 |
| 4/96 | | | | |
| DOTAP/DOPE = | 20 | +23.6 | +17.0 | 150 |
| 20/80 | | | | |
| DOTAP/DOPC = | 30 | +46.3 | +27.0 | 137 |
| 30/70 | | | | |
| DOTAP/DOPE = | 40 | | +33.1 | 140 |
| 40/60 | | | | |
| DOTAP/DOPE = | 55 | +50.3 | +42.9 | 151 |
| 55/45 | | | | |
| DOTAP/DOPE = | 80 | +51.4 | +43.0 | 130-135 |
| 80/20 | | | | |
| DOTAP | 100 | | +48.9 | 140 |

Sample conductivity approximately 3 mS/cm² (pH 6.8) and 1 mS/cm² (pH 8), respectively.

TABLE 3
Measurement of Seven Liposome Formulations
(varying content of DOTAP, co-lipid DOPC)
in 0.05 M solution of KCl (pH 7.0 to 7.5).
Sample conductivity approximately 2.65 mS/cm².

| | DOTAP | Zeta potential in |
|---|---|---|
| | concentration | mV in 0.05 M KCl, |
| Liposome formulation | [mol %] | pH 7.0 to 7.5 |
| DOTAP/DOPC = 4/96 | 4 | +16.6 |
| DOTAP/DOPC = 15/85 | 15 | +39.6 |
| DOTAP/DOPC = 30/70 | 30 | +49.0 |
| DOTAP/DOPC = 50/50 | 50 | +59.9 |
| DOTAP/DOPC = 55/45 | 55 | +58.4 |
| DOTAP/DOPC = 80/20 | 80 | +59.4 |
| DOTAP | 96 | +61.5 |

These results indicate that for DOTAP concentrations between 4 and approximately 50 mol %, there is a constant increase in Zeta potential. Surprisingly however, for DOTAP concentrations of 50 mol % and higher, the Zeta potential levels off and reaches values between +40 and +62 mV, overall exhibiting a hyperbolic curve shape. The data demonstrate that this hyperbolic shape of the curve is maintained in different buffer systems, even though the absolute values for zeta potential change slightly. While the curve in KOH/HCl (pH 7.5) represents the most physiological situation, the other two curves at pH 6.8 and 8.0 illustrate that even with some deviation from physiological pH, the shape of the curve is maintained.

In this example, DOTAP is used for measurement of zeta potential in liposome formulation. It is within the skill of the artisan to substitute other cationic lipids for DOTAP and to measure the zeta potential of the liposome formulation.

### Example 5

### Selective Targeting of Positively Charged Molecules to Angiogenic Endothelial Cells In Vivo

The transport and specific interactions of macromolecules to tumor tissue are dependent upon different parameters, e.g., on the size and the charge of the molecules. This example shows charge dependent targeting to angiogenic endothelial cells in vivo. Fluorescently labeled charged dextrans from Molecular Probes or synthesized using methods described below are used. The indirect tumor targeting of those charged molecules to angiogenic endothelial cells is shown using a hamster chamber model (Endrich et al, 1980).

1. Fluorescently Labeled Dextrans with Different Net Charge.

Dextrans—hydrophilic polysaccharides—are characterized by their high molecular weight, good water solubility, low toxicity and relative inertness. These properties make dextrans effective water soluble carriers for dyes, e.g., fluorescent dyes. Their biologically uncommon α-1,6-polyglucose linkages are resistant to cleavage by most endogenous cellular glycosidases.

a. Fluorescently Labeled Dextrans from Molecular Probes

To analyze the behavior of dextrans carrying a positive net charge, cationic fluorescently labeled dextrans from Molecular Probes were used. Examples of such dextrans are Rhodamine Green™ coupled dextrans having molecular weights varying between 3,000 and 70,000 or lysine conjugated, tetramethylrhodamine coupled dextrans, which in spite of the anionic group coupled to each dextran molecule has a positive net charge mediated by the conjugated cationic lysine residues. It is also possible to use other fluorescent dextrans, which contain lysine residues resulting in a positive net charge.

For fluorescent dextrans with a negative net charge, fluorescent anionic or polyanionic dextrans from Molecular Probes without any lysine residues were used. Examples of anionic or polyanionic dextrans include Cascade Blue® coupled dextran, with molecular weights between 3,000 to 70,000 or Fluorescein coupled dextrans or negative charged dextrans carrying other fluorescent labels.

For neutral dextrans with a net charge of zero, Molecular Probes' dextrans such as rhodamine B coupled neutral dextrans with molecular weights in the range from 10,000 to 70,000 or other fluorescent neutral dextrans were used.

b. Synthesis of Charged or Neutral Fluorescently Labeled Dextrans

Charged or neutral unlabeled dextrans were synthesized by oxidation of dextran molecules (for example by periodate) producing reactive aldehyde groups. Subsequently, the aldehyde groups were reacted with the amine function of various reagents producing molecules of different net charge. Finally, those molecules were conjugated to fluorescent dyes.

i) Oxidation of Dextrans

Unlabeled dextran molecules were mixed with sodium periodate in an appropriate molar ratio up to 6 mol dextrose to 1 mol IO₄⁻ in aqueous solution (30 min, pH 6). The reaction was stopped by adding ethyleneglycol (about 300 fold molar excess) and was dialyzed against 0.15 mol NaCl.

ii) Preparation of Positively Charged Fluorescently Labeled Dextrans

The modified oxidized dextrans were mixed with an aqueous solution of polylysine in a sodium borate buffer (pH˜9) in the appropriate way. The resulting solution was dialyzed against 0.15 M NaCl. Subsequently, the remaining primary amino groups of the polylysine were reacted in 0.1 M sodium carbonate buffer with the respective succinimidyl ester or sulfonyl chloride of a fluorescent dye, e.g., a member of the Cy-Dye family from Amersham or a Fluoresceine derivative or any other reactive dyes. The chosen molar ratio of fluorophore to polylysine-dextran was determined ahead of time to give the resulting reaction product a positive net charge. Free dye was separated by dialyzing the sample against 0.15 M NaCl. The isoelectric point of the reaction product was determined by isoelectric focusing in physiological buffer to be above 8.

iii) Preparation of Fluorescent Neutral Dextrans

Oxidized dextrans were reacted with an appropriate peptide carrying two primary amino groups, e.g., alanine-alanine-lysine, using the free amino group of the peptide backbone for coupling to dextran. Subsequently, 0.1-10 mol % of the primary amino groups of the polylysine were reacted with the sulfonyl chloride or the succinimidyl ester of a fluorescent dye, for example Lissamine Rhodamin B, Fluorescein derivatives or any other reactive fluorescent dyes, resulting in a molecule carrying a zero net charge. The isoelectric point of the reaction product was determined by isoelectric focusing in physiological buffer to be between 7 and 7.5.

iv) Preparation of Fluorescent Negatively Charged Dextrans

Oxidized dextrans were reacted with an appropriate peptide consisting of negatively and positively charged amino acids having a net charge of zero or lower, e.g., glutamate-glutamate-lysine. As described above, the free amino groups of the peptide's N-terminus were conjugated to the aldehyde groups of the oxidized dextran. 0.1-10 mol % of the aliphatic amino groups of the lysine residues were then reacted to an appropriate reactive fluorescent dye conjugate (see above). The isoelectric point of the reaction product is checked by isoelectric focusing in a physiological buffer and was typically found to be below 5.

2. Targeting Fluorescently Labeled Dextrans to Angiogenic Endothelial Cells in vivo

Male Syrian golden hamsters (40-50 g body weight) were fitted with titanium dorsal skinfold chambers. Chamber preparation was performed under pentobarbital anaesthesia (50 mg kg⁻¹ intraperitoneal). Following implantation of the transparent access chamber and a recovery period of 24 h from anaesthesia and microsurgery, only preparations fulfilling the criteria of microscopically intact microcirculation were utilized for implantation of 2×10⁵ cells of the amelanotic melanoma (A-Mel-3) of the hamster (Fortner et al., 1961) into the chamber. In the present tumor model, angiogenesis has been well characterized. The experiments were performed after 6-7 days of tumor growth, when functioning tumor microcirculation was established. An appropriate amount of fluorescently labeled charged dextrans was injected via an indwelling fine polyethylene catheter in the right jugular vein implanted 24 h before injection of the sample. During the experiment, the awake chamber bearing hamster was immobilized using a Perspex tube on an especially designed stage. The tumor endothelium specific homing of the fluorescent dextrans was analyzed by fluorescence microscopy of angiogenic tumor tissue and surrounding host tissue at different time points after injection (0.5-360 min). The fluorescence intensities in both tissue types were determined as percent of a reference fluorescence signal (% standard) present in each chamber. The ratio of % standard fluorescence in tumor and surrounding tissue, which is defined as the selectivity of a substance for tumor tissue, is the a value for the affinity with which charged dextrans bind to the tumor endothelium and is shown in FIG. 4.

3. Conclusion

In general, the binding affinity of charged dextrans to angiogenic tumor endothelium increases with the positive net charge of the delivered molecules (see FIGS. 4A-C). This indicates that cationic macromolecules adhere to the negatively charged binding sites located on the cell surface or the glycocalyx of the tumor endothelium.

### Example 6

### Vascular Tumor Targeting in a Human Tumor Xenograft: Molecular Charge Dependence

As noted earlier, molecular charge is one of the properties that affect transport across a blood vessel wall. However, there are no data available on the effect of molecular charge on microvascular permeability for macromolecules following higher accumulation in angiogenic tumor vessels in solid tumors. Thus, in this example, tumor microvascular permeability for different proteins having similar size but different charge was measured. Measurements were performed in the human colon adenocarcinoma LS174T transplanted in transparent dorsal skinfold chambers in severe combined immunodeficient (SCID) mice. Bovine serum albumin (BSA) and IgG were fluorescently labeled and were either cationized by conjugations with hexamethylenediamine or anionized by succinylation. The molecules were injected I.V. and the fluorescence in tumor tissue was quantified by intravital fluorescence microscopy. The fluorescence intensity and pharmacokinetic data were used to calculate the microvascular permeability.

1. Animals and Tumor Model

Dorsal skinfold chambers bearing the LS174T tumor were prepared in male severe combined immunodeficient (SCID) mice as described earlier (Leunig et al., 1992). In brief, titanium chambers were implanted in the dorsal skin of mice (male, 6-8 weeks old, 25-35 g) under anesthesia (75 mg ketamine hydrochloride and 25 mg xylazine per kg body weight subcutaneously). Two days later, 2 μl of a dense suspension of human colon carcinoma cells (approx. 2×10⁵ cells in phosphate buffered saline) were inoculated onto the striated muscle layer of the subcutaneous tissue in chambers. Experiments for measurement of permeability were performed 2 weeks after tumor cell implantation.

2. Preparation of Charge-Modified BSA

Bovine serum albumin (BSA; A7030; Sigma Chemical Co., St. Louis, Mo.) was first fluorescently labeled by conjugation with carboxytetramethylrhodamine succimidyl ester (C-1171; Molecular Probes, Eugene, Oreg.). Free dye was removed on a gel filtration column (Econo-Pac 10DG; Bio-Rad Laboratories, Herculaes, Calif.) equilibrated with 50 mM phosphate buffered saline (PBS; Sigma) containing 0.002% sodium azide (Sigma). This procedure yielded a molar dye/protein ratio of 1.3. Subsequently, aliquots of the BSA-solution were anionized by succinylation, or cationized by conjugation with hexamethylenediamine. For succinylation (Klotz, 1967; Rennke & Venkatchalam, 1978), 10 mg of succinic anhydride (Sigma) in 50 ml of dimethylsulfoxide (DMSO, Sigma) was added dropwise in small increments to the solution of 10 mg protein in 1 ml of 0.2 M bicarbonate buffer, pH 8.0. During the incubation time (30 min. at room temperature) pH was maintained at 8.0 to 8.5. For cationization (Triguero et al., 1989; Kumagai et al., 1987; Hoare & Koshland, 1967), free carboxylic groups of the protein (10 mg in 1 ml of 5 mM MES, pH 5.3, if necessary, pH was maintained at 5.3 with 1 M HCl) were activated with 1-ethyl-3-(3-dimethylaminopropyl)carboxiimide (CDI, Sigma). For this purpose, two portions of the CDI (each 10 mg CDI in 100 ml of water) were added to the protein solution in 10 min intervals. Immediately after the addition of the second portion of CDI, the activated protein was added upon stirring into 2 ml of 2 M hexamethylenediamine (Sigma) in water, pH of the solution was adjusted to 6.8 and the mixture was incubated for 5 hours at room temperature. After conjugation, the products were purified by overnight dialysis, and high molecular weight aggregates were removed by elution through a Sepharose CL-6B column (Pharmacia). The major protein peak was pooled and stored at 4° C.

3. Preparation of Charge Modified IgG

The solvent of mouse monoclonal IgG antibody (MOPC21; M-9269, Sigma) was exchanged to 0.2 M sodium bicarbonate buffer on a gel filtration column (Econo-Pac 10DG; Bio-Rad) and the solution concentrated to 1 mg protein /ml by centrifugal filtration (Ultrafree-CL; Millipore, Bedford, Mass.). pH was adjusted to 9.3. For fluorescent labeling, Cyanine 3 monofunctional dye (Cy3-Mono-OSu; PA13104; Amersham Life Science, Arlington Heights, Ill.) was used to yield high fluorescence intensity, which allowed in vivo experiments with small amounts of IgG (0.5 mg IgG/animal). Cy3-Mono-OSu was added at a ratio of 2 mg dye/10 mg protein and the solution stirred at room temperature for 60 min. Free dye was removed on a gel filtration column (Econo-Pac IODG; Bio-Rad) equilibrated with 50 mM PBS containing 0.002% sodium azide (Sigma), and the solution concentrated to 1.8 mg protein/ml by centrifugal filtration (Ultrafree-CL; Millipore). Subsequently, anionic and cationic derivatives of IgG were prepared as described above for BSA.

4. Measurement of Molecular Charge and Weight

Isoelectric points of the proteins were determined by isoelectric focusing using polyacrylamide gel slabs on a vertical electrofocusing apparatus. pl was quantified by comparison with protein standards (Bio-Rad) after Coomassie Blue staining of the gels. The molecular weights of the proteins were analyzed by SDS-PAGE (Mini-Protean II; Bio-Rad) with and without reducing agent P-mercaptoethanol in the sample buffer.

5. Measurement of Tumor Microvascular Permeability

Effective microvascular permeability to the different proteins was measured using fluorescence microscopy as reported previously (Yuan et al., 1993; 1995). In brief, animals were anesthetized as described above and immobilized in a polycarbonate tube on the microscope stage. The fluorescently labeled protein was dissolved in PBS and injected into the tail vein as a bolus (0.1 ml/25 g body weight). Fluorescence intensity of tumor tissue was depicted by a 20x objective of a fluorescence microscope (Axioplan, Zeiss), and quantified over a period of 20 min by a photomultiplier. Off-line analysis from the videotaped tumor area allowed measurement of tumor vascular surface and volume. In separate experiments using three animals for evaluation of the behavior of each protein, the time constant of plasma clearance was quantified after collection of arterial blood samples within 30 min following injection of the protein. Tumor microvascular permeability from experiments with 6 to 7 individual tumors for each of the different proteins was calculated from these data according to Yuan et al. (Yuan et al., 1993). Results for microvascular permeability are given as median±standard error of median.

The results are shown in Table 4, below.

TABLE 4
Characteristics of Tracer Molecules and their Microvascular
Permeability in Tumors.
Anionized and cationized BSA and IgG were prepared from the
native proteins as described in materials and methods.

| | | | Median K | Median Pᵥ |
|---|---|---|---|---|
| | | pI^{a} | (range) | (range) |
| Name | Mᵣ | (range) | (100s) | (10⁻⁷ cm/s) |
| Native BSA^{b} | 66,000 | 4.5 | 80.5 | 1.61 |
| | | | (77-130) | (0.65-1.93) |
| Anionized BSA | 64,000 | ≈2.0 | 40.8 | 1.11 |
| | | | (37-45) | (0.95-2.38) |
| Cationized BSA | 64,000 | 8.6-9.1 | 12.7 | 4.25 |
| | | | (12-13) | (3.57-5.34) |
| Native IgG^{b} | 160,000 | 6.0 | 50.0 | 2.82 |
| | | | (34-82) | (1.47-4.07) |
| Anionized IgG | 155,000 | 3.0-3.9 | 39.3 | 1.93 |
| | | | (37-44) | (1.11-2.53) |
| Cationized IgG | 155,000 | 8.6-9.3 | 11.0 | 4.65 |
| | | | (5-15) | (4.25-5.27) |

^{a}pI, isoelectric point; K, time constant of concentration decay in the plasma; Pᵥ, microvascular permeability.
^{b}Data from Yuan et al. (1995).

6. Conclusion

Fluorescence microscopy revealed that the different proteins extravasated homogeneously along the vessel walls. Tumor microvascular permeability (Pv) to anionized and cationized BSA is shown in Table 4. Permeability values were low for negatively charged BSA (Pv=1.11±0.44×10⁻⁷ cm/s; median±standard error of median), and almost 4-fold higher to positively charged BSA (Pv=4.25±0.26×10⁻⁷ cm/s).

The microvascular permeability of tumors to anionized and cationized IgG was slightly above values measured for corresponding BSA samples (Table 4). Similar to the data for charge-modified BSA, cationized IgG revealed highest permeability (Pv=4.65±0.29×10⁻⁷ cm/s), and permeability of anionized IgG (Pv=1.93±0.41×10⁻⁷ cm/s) was below the value measured for the native protein.

### Example 7

### Uptake of Neutral and Cationic Rhodamine-labeled Liposomes by Human Endothelial Cell Cultures (aUVEC)

HUVEC were seeded at cell densities of 2×10⁴ cells/cm² in gelatin coated 24-well culture plates and cultured for 48 h in endothelial growth medium with 2% fetal calf serum at 37° C. and 5% CO₂ in a humidified atmosphere. The culture medium was removed, cells were washed with PBS and 500 iIl of serum free endothelial basal medium was added. Rhodamine-labeled liposomes (0.5 mM Rhodamine label) were added to the cultures at a concentration of 100 μM total lipid. After 4 h of cultivation the medium was removed and the cultures were washed twice with 500 μl PBS. The cells were lysed with 1.5 ml 1% Triton X-100 in PBS for 30 min at room temperature. Fluorescence intensity was measured at an excitation wavelength of 560 nm and an emission wavelength of 580 run in a SPEX FluoroMax-2.

The results of this experiment are shown in Table 5, below.

TABLE 5
Uptake of Rhodamine-Labeled Liposomes by HUVEC (total lipid
concentrations 10 mM, liposome compositions are reported in mol %)
Zeta potentials were measured under the conditions specified for Table 3.

| | | | | | | |
|---|---|---|---|---|---|---|
| DOTAP | 0 | 30 | 40 | 50 | 80 | 95 |
| DOPC | 55 | 65 | 55 | 45 | 15 | 0 |
| Chol | 40 | 0 | 0 | 0 | 0 | 0 |
| Rh-DOPE | 5 | 5 | 5 | 5 | 5 | 5 |
| Zeta | — | +27.0 | +33.1 | +42.9 | +43.0 | +48.8 |
| Potential (mV) | | | | | | |
| Fluores-cence | — | 124802 | 179961 | 230879 | 256276 | 271732 |
| Uptake (cps) | | | | | | |

### Example 8

### Preparation of a Liposomal, Cationic Imaging Agent

i) Preparation of the Imaging Agent

Cellular imaging agents are encapsulated into cationic liposomes comprising cationic lipid, e.g., DOTAP. For example, magnetite (Fe₃O₄) is known in the art to be encapsulated in cationic liposomes for imaging or treating of hyperthermia, respectively. The iron oxides can be entrapped within the interior of the cationic liposomes by following the general methods described above or, for example, the method described in U.S. Pat. No. 5,088,499. For the treatment of hyperthermia, iron oxide particles are administered intravenously to a cancer patient. The particles accumulate in the tumor. When the patient is put into a magnetic field, the iron oxide particles are heated and consequently, destroy the solid tumor.

As a specific example, superparamagnetic iron oxide particles which were stabilized electrostatically by H⁺ ions (commercially available from Berlin Heat AG) are encapsulated in liposomes comprising DOTAP and DOPC at a ratio of 50/50 and with a total lipid concentration of 15 mM initially. Such a formulation is prepared as follows: DOTAP (0.075 mmol) and DOPC (0.075 mmol) are dissolved in 20 ml of chloroform and placed in a 500 ml round bottom flask. The chloroform is evaporated under vacuum and the film is dried at a reduced pressure of 5 mbar for 90 minutes. Subsequently, the lipid film is rehydrated with 10 ml of an aqueous solution of iron oxide particles having a concentration of 286 mM. The liposomal suspension is mixed gently and stored in the refrigerator. After 24 hours, the suspension is centrifuged at 12,000 G at 10° C. for 30 minutes. This yields a separation of the mixture into two phases: an upper phase, containing a large portion of the liposomes with encapsulated iron oxide and a lower phase depleted of liposomes but containing nonencapsulated iron oxide. The upper fraction (10 ml) is extruded (Lipex extruder, barrel with volume of 10 ml) five times through a 400 nm polycarbonate membrane (Osmoics Inc.). The extruded product was analyzed for lipids by HPLC and for iron photometrically (thiocyanate method).

ii) Application of the Imaging Agent

For animal studies, C57BL/6 mice were inoculated with 106 Lewis Lung Carcinoma (LLC) cells. Approximately 10 days after inoculation, the mice developed palpable tumors. When the tumor reached a size of approximately 5-8 nm (measured in two dimensions), the animal is placed into a 2T MR tomograph (Bruker), anesthetized (isofluran inhalation) and scanned for anatomical orientation. During this scan, T1 and T2 relaxivities were recorded for later comparison. Subsequently, 14 μl of the imaging agent formulation (prepared as described above) per gram animal weight were injected into the tail vene. The mouse was repositioned into the tomograph and scanned at various time points after injection. Table 6 summarizes the relaxivity data measured in a representative experiment in the tumor of an animal which received the above described formulation. The T2 relaxivity of the normal tissue (e.g., muscle) did not change (data not shown).

TABLE 6
T2 Values in Tumor Tissue Before and After Application
of the Liposomal Cationic Contrast Agent

| T2 before | | | |
|---|---|---|---|
| application of | T2 after application of contrast agent | | |
| contrast agent | T2 after 15 min | T2 after 60 min | T2 after 4 hours |
| 86.8 ms | 81 ms (93% of | 75 ms (86% of | 68 ms (78% of |
| | initial T2) | initial T2) | initial T2) |

### Example 9

### Magnetosomes as Cationic Imaging Agents

Magnetosomes are composed of a nanometer-sized magnetite core, which is enwrapped by a lipid layer. The preparation of magnetosomes with a cationic outer layer can occur in a similar manner as described for using negatively charged or neutral phospholipids (De Cuyper et al., 1990). The in vivo testing of the magnetosomes was carried out in C57BL/6 mice which had been inoculated with 10⁶ Lewis Lung carcinoma cells. For imaging purposes, T2 relaxivities of several tissues were measured by MR.

1. Preparation of positively charged Magnetosomes

Magnetite cores surrounded by a lipid layer can be prepared for example by replacement of the lauric acid monolayer of iron oxide particles by lipids. The exchange of the layer occurs spontaneously upon incubation of the lauric acid coated magnetite particles with liposomes composed of 30-70 mol % of a phospholipid and of 70 to 30 mol % of a cationic lipid. It is assumed that the phospholipid binds to the oxygen atoms in Fe₃O₄ and thus replaces lauric acid. The cationic component accumulates preferentially in the outer layer of the magnetomsome and stabilizes them electrostatically. The excess, lauric acid is dialyzed from the mixture. The product is subsequently purified.

As a specific example, 150 μl of a suspension containing lauric acid coated superparamagnetic iron oxide particles (Fe concentration 2 M) were incubated at 37° C. with 10 ml of a 10 mM liposomes formulation comprising DOTAP and DOPC (Avanti Polar Lipids, Inc., Alabaster) at a molar ratio of 30/70 mol % (synthesized as described in example 4). Subsequently, the mixture was dialyzed for 5 days against 5% glucose solution. The depletion of the lauric acid during the dialysis process was monitored after derivatization of the lauric acid with phenacylbromide (Borch et al., 1975) by HPLC (LiChrospher RP-select B 5 μm 250-4 (Merck), acetonitrile/water 75:25, flow=1 ml/min, A=254 m-n, k′=3.0, k′=(tr-to)/to).

The unencapsulated iron oxide particles were separated from the magnetosomes and the excess empty liposomes by gel chromatography on Sephacryl S-300 HR. The magnetosomes were separated from the empty liposomes on superparamagnetic MACS microbeads using a strong permanent magnet (Miltenyi Biotec GmbH, Bergisch Gladbach). Table 7 summarizes the analytics of the magnetosomes.

TABLE 7
Analytical Data of Magnetosomes Measured Before and After
Purification of the Particles.

| Magnetosomes | Total | | particle size | | |
|---|---|---|---|---|---|
| lipid | lipid | | (nm) | Polydis- | Zeta |
| concentration | in | Fe in | measured as | persity | potential |
| in mol % | mM | mM | Zₐᵥₑ | index | (mv) |
| DOTAP/DOPC 30:70 | 3.83 | 11.4 | 201.2 | 0.3 | n.d. |
| mol % after incubation | | | | | |
| and dialysis | | | | | |
| DOTAP/DOPC 30:70 | 1.3 | 13.9 | 216.6 | 0.3 | +41.5 |
| mol % after | | | | | |
| purification (gel | | | | | |
| chromatography, | | | | | |
| MACS microbeads) | | | | | |

2. Imaging of C57BL/6 Mice with a Lewis Lung Carcinoma

For animal studies, C57BL/6 mice were inoculated with LLC cells (approx. 10⁶ cells in phosphate buffered saline) subcutaneously. Approximately 10 days after inoculation, the mice developed palpable tumors. When the tumor reached a size of about 5-8 mm (measured in two dimensions), the animal was anesthetized (isofularan inhalation), placed into a 2 T MR tomograph (Burker) on a thermostated pad, and scanned for anatomical orientation. During this scan, T1 and T2 relaxivities were recorded for later comparison. Subsequently, 14 μl of the imaging agent formulation (prepared as described above) per gram animal weight were injected into the tail vein. The mouse was repositioned into the tomograph and scanned at various time points after injection. Table 8 summarizes the relaxivity data measured in various animals which received the above described formulation.

TABLE 8
T2 Values in Tumor Tissue of Representative Animal Experiments
Before and After Application of the Cationic Magnetosomes

| | T2 before | | |
|---|---|---|---|
| | application of | T2 after application of contrast agent | |
| Formulation | contrast agent | T2 after 30 min | T2 after 3.5 hours |
| DOTAP/DOPC | 82 ms | 65 ms | 72 ms |
| 30:70 mol % | | (79% of | (88% of |
| after dialysis | | initial T2) | initial T2) |
| DOTAP/DOPC | 83 ms | 71 ms | 69 ms |
| 30:70 mol % | | (86% of | (83% of |
| after purification | | initial T2) | initial T2) |

### Example 10

### Cationic Microemulsions Containing the Lipophilic Drug Paclitaxel as Carrier for Water Insoluble Drugs

Stable oil-in-water (O/W) emulsions as suitable carriers for lipophilic drugs (e.g. paclitaxel) were obtained by homogenization with an electrical stirrer or sonicator (Tuchida et al., 1992, Cavalli et al., 2000). The oil phase composed of several lipids acts as a solubilizer for approximately 2.1 mol % of the drug preventing its crystallization for several month. With regard to in vivo applications the main components of the hydrophobic matrix were chosen to be biocompatible and biodegradable lipids like triglycerides (TG). For targeting purposes only up to 5 mol % DOTAP or DDAB are required as cationic emulsifiers, corresponding to 50% of cationic amphiphile in the outer layer. The particle size is affected by the weight ratio of lipophilics (TG) to amphiphilics, (TG/A) and is correlated with increasing amounts of TG.

Paclitaxel (10 mg) was dissolved in 560 mg of Trioctadecylglyceride. Next, a lipid mixture comprising 25 mg DOTAP, 25 mg DOPC (ration TG/A=11) was dispersed in the TG/paclitaxel mixture by homogenizing (IKA Ultra-Turrax T8, 10000 rpm) at room temperature for 10 minutes. Then 7 ml of a 5% glucose solution was added dropwise to the oil-lipid mixture under continuing homogenization for 15 minutes. The data in Table 9 illustrate that a principle of cationization can be equally applied to microemulsions with or without drugs and results a stable formulation with sufficiently high zeta potential for angiogenesis targeting. With this approach, a high ratio of drug to cationic component can be achieved (here: 1:2.5 weight %), resulting in a significant improvement of tolerability of the drug delivery system.

TABLE 9
Analytical Data of Cationic Microemulsions Composed of
Triglyceride (TG), DOTAP, DOPC and Paclitaxel After Centrifugation
(500 g, 10 min).

| Cationic | | | Paclit- | Ratio TG/ | Zₐᵥₑ | | Zeta |
|---|---|---|---|---|---|---|---|
| lipid | DOPC | TG | axel | Amphi- | in | | potential |
| [mg] | [mg] | [mg] | [mg] | phile | nm | PI | in mV |
| DOTAP: | 25 | 560 | 0 | 11 | 253 | 0.3 | +60.9 |
| 25 | | | | | | | |
| DOTAP: | 25 | 560 | 10 | 11 | 295 | 0.4 | +56.3 |
| 25 | | | | | | | |
| DDAB: | 28 | 560 | 3 | 10 | 298 | 0.4 | +58.8 |
| 28 | | | | | | | |

### Example 11

### Preparation of Other Encapsulated Imaging Agents

Representative imaging agent formulations include cationic liposomes with encapsulated liposomal magnetite particles (as described in Example 8), cationic liposomes wherein magnetite particles are covalently attached to lipid bilayer, cationic liposomes with Gd-DTPA, encapsulated Gd-complexes, cationic liposomes with Gd covalently attached to lipid bilayer, cationic liposomes with X-ray attenuating complexes/molecules either inside encapsulated, or attached to membrane or both for CT or X-ray imaging studies. By using known techniques, a representative number of which are identified below, and preparing formulations to achieve the zeta potential ranges described above, the skilled artisan should be able to formulate and administer a wide variety of imaging agents.

Techniques for encapsulation of Gd-DTPA are well known to the skilled artisan (see Unger, E. C., P. MacDougall, P. Cullis and C. Tilcock, “Liposomal Gd-DTPA: effect of encapsulation on enhancement of hepatoma model by MRI.” Magnetic Resonance Imaging 7:417-23. (1989)).

U.S. Pat. No. 6,001,333 describes a method of preparing a liposomal contrast agent for detection of tumors by CT imaging. The method comprises the following steps: a) mixing maltose with water in the ratio of about 20 grams maltose to 100 ml of water and stirring until the maltose is dissolved to form an aqueous solution; b) mixing egg phosphatidylcholine with 99.6% ethanol in the ratio of about 4.2 g of the egg phosphatidylcholine to 5 ml of the ethanol and stirring until dissolved to form an alcohol solution; c) adding BHT to the aqueous solution in the ratio of about 6.2 mg BHT to 20 g maltose; d) adding the alcohol solution to the aqueous solution in a dropwise manner with continuous mixing until a solution is obtained which contains 5 ml ethanol for each 450 ml of water to form an encapsulating solution; e) stirring the substance to be encapsulated into the encapsulating solution; f) passing the mixture from step e above through a microfluidizing device to form a clear solution; g) lyophilizing the mixture from step f.

It is within the skill of the artisan to modify the above method by replacing an appropriate amount of the egg phosphatidylcholine with cationic lipid to obtain a liposomal composition containing the agent and having the desired zeta potential and/or isoelectric point, so that the agent will selectively target the tumor.

Other methods for preparation of liposomal formulations are well known to the skilled artisan These include but are not limited to hydration of lipid films, solvent injection, reverse-phase evaporation, and a combination of these methods with freeze-thaw cycles. It is also within the skill of the artisan to prepare liposomes by sonication, pH induced vesiculation, or detergent solubilization. Moreover, various methods are also available for separation of encapsulated and nonencapsulated molecules including but not limited to gel filtration, ultracentrifugation, cross-flow filtration, density gradient centrifugation, and dialysis.

### Example 12

### Tumor Regression in Nude Mice.

liposomal composition generally formulated according to Example 4 to contain diphtheria toxin is injected into tumor-bearing nude test mice. Parallel injections into control tumor bearing nude mice are made with a similar composition not derivatized in order to modulate its zeta potential. After two rounds of injections at two day intervals, the test and control mice are sacrificed fourteen days post injection and examined by dissection. The test mice display statistically significant decreases in tumor mass, showing that the composition was therapeutically effective to cause tumor regression.

Other compositions that are useful for causing tumor regression include liposomal composition comprising paclitaxel, docetaxel, or other taxanes, vincristine, navelbine, and other vinca alkaloids, gemcitabine and other nucleoside analogs, cisplatinum and other platinum compounds. These compositions can be formulated according to Example 4.

### Example 13

### Tumor Imaging of a Bladder Tumor in Cancer Patients

fluorescent imaging agent was prepared according to the protocol described in Example 4 and was administered systemically to a cancer patient with bladder tumor (urothelium carcinoma). The applied fluorescent imaging agent was formulated as a liposomal suspension containing 50 mol % DOTAP, 45 mol % DOPC and 5 mol % rhodamine-DOPE in 5% glucose and a total lipid content of 10 mM. The formulation was applied systemically to the patient in a dose of 0.5 mg total lipid per kilogram body weight using an infusion rate of 2 ml/min.

During and following the treatment the accumulation of the fluorescent imaging formulation was detected using a conventional endoscope for bladder surgery fitted with a fluorescent filter set specific for the liposomal fluorescent dye. The accumulation of fluorescent dye in the tumor tissue was visualized by imaging as well as by spectroscopic identification of the dye. Due to fluorescent labeling of the tumor edges, the tumor tissue could be clearly discriminated from normal bladder epithelium, and the tumor was excised completely.

### Example 14

### Imaging of Cancer Patients with Solid Tumors

A MRI imaging agent is prepared generally according to the protocols described in Examples 4 and 8 and administered to a cancer patient. The MRI imaging agent is formulated into a liposome formulation containing 40 mol % Dotap, 60 mol % DOPC (total lipid concentration 40 mM) and al Fe concentration of 9 mM. Ten ml of the formulation is administered to a 80 kg patient which is approximately 5 mg Fe per patient or about 0.06 mg Fe/kg body weight (about 10% of currently administered amount of Fe).

### Example 15

### Treatment of Cancer Patients with Solid Tumors

The therapeutic agent prepared as shown in Example 9 is prepared and administered to a human patient for tumor treatment. Therapeutically effective amounts of the formulation are administered intravenously to a patient suffering from one or more solid tumor growths. Therapy is maintained until tumor regression has occurred as determined by one or more markers of regression, including a decline in circulating tumor antigens and/or physical resorption. Subsequent continuous or periodic treatments with the formulation are optionally indicated as a prophylactic or as a means to ensure total tumor regression.

### Example 16

### Treatment of Patients with Retrolenta Fibroplasia.

A patient suffering from retrolenta fibroplasia is treated with cryotherapeutic ablation. In addition, a therapeutic formulation as described in Example I 1 also is administered to the patient. Revascularization of the ablated area is reduced or prevented.

### Example 17

### Combination Therapy.

A patient suffering from one or more solid tumors is treated according to Example 11. Following the initial course of therapy, the patient is subjected to traditional chemotherapy and/or radiation therapy. Therapeutic progress is monitored as required by general oncology protocols. Use of the combination therapy permits reduced exposure of the patient to radiation or chemotherapeutics.

### Example 18

### Coadministration of a Therapeutic Composition with a Second Active Ingredient.

A liposomal formulation as described in Example 11 is co-formulated with an immunotoxin as described in Thorpe et al. U.S. Pat. No. 5,965,132. Therapeutically effective amounts of co-formulated liposomes are administered to a patient suffering from one or more solid tumors. Tumor regression is observed.

### Example 19

### Wound Healing

A liposomal composition as described in U.S. Pat. No. 5,879,713 comprising bFGF or VEGF is formulated for promoting would healing in a patient. Therapeutically effective amount of bFGF or VEGF is added to the liposomal composition comprising DOTAP:DOPC (40:60). Therapeutic effective amounts of the liposome formulation are administered to a patient in need of wound healing. Wound healing is observed.

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents and patent applications that are identified in this patent application are incorporated by reference in their entirety.

1. A method of selectively targeting a composition, selected from the group of: (a) particles, excluding liposomes, having a zeta potential in the range of about +25 mV to +100 mV in about 0.05 mM KCl solution at about pH 7.5; (b) molecules having an isoelectric point above 7.5; (c) liposomes containing cationic lipids in the range of about 25 mol % to 50 mol %; (d) magnetosomes with a cationic lipid layer having a zeta potential in the range of about +25 to +100 mV in about 0.05 mM KCI solution at about pH 7.5 and (e) oil-in-water emulsions or microemulsions containing cationic amphiphiles in the outer layer in the range of about 25 to 60 mol % or having a zeta potential in the range of about +25 mV to +100 mV in about 0.05 mM KCl solution at about pH 7.5.

2. The method of claim 1, wherein the activated vascular site is selected from the group consisting of: (a) sites of angiogenesis; (b) sites of inflammation; (c) sites of wound healing; and (d) the blood brain barrier.

3. An imaging composition for selective targeting to an activated vascular site, comprising an imaging agent and a carrier selected from the group consisting of: (a) particles, excluding liposomes, having a zeta potential in the range of about +25 mV to +100 mV in about 0.05 mM KCl solution at about pH 7.5; (b) molecules having an isoelectric point above 7.5; and (c) liposomes containing cationic lipids in the range of about 25 mol % to 50 mol %; (d) magnetosomes with a cationic lipid layer having a zeta potential in the range of about +25 to +100 mV in about 0.05 mM KCl solution at about pH 7.5 and (e) oil-in-water emulsions or microemulsions containing cationic amphiphiles in the outer layer in the range of about 25 to 60 mol % or having a zeta potential in the range of about +25 mV to +100 mV in about 0.05 mM KCl solution at about pH 7.5.

4. The imaging composition of claim 3, wherein the imaging agent is selected from the group consisting of iron oxide particles, dyes, fluorescent dyes, NMR labels, scintigraphic labels, gold particles, PET labels, ultrasound contrast media, and CT contrast media.

5. A method of selectively targeting an imaging agent to a site of angiogenesis in an animal, comprising the steps of administering to the animal a composition according to claim 3 or 4, and allowing the composition to selectively accumulate to a diagnostically effective level in the vicinity of the site of angiogenesis.

6. The method of claim 5, wherein the composition is administered by a route selected from the group consisting of oral administration, intravenous administration, transdermal administration, subcutaneous administration, intraperitoneal administration, intratumoral administration, intraarterial administration, intramuscular administration, instillation and aerosol administration.

7. A therapeutic composition for selective targeting to an activated vascular site, comprising a therapeutically effective amount of an active ingredient and a carrier, the composition being selected from the group consisting of. (a) particles, excluding liposomes, having a zeta potential in the range of about +25 mV to +100 mV in about 0.05 mM KCl solution at about pH 7.5; (b) molecules having an isoelectric point above 7.5; (c) liposomes containing cationic lipids in the range of about 25 mol % to 50 mol %; (d) magneto somes with a cationic lipid layer having a zeta potential in the range of about +25 mV to +100 mV in about 0.05 mM KCl solution at about pH 7.5; (e) oil-in-water emulsions or microemulsions containing cationic amphiphiles in the outer layer in the range of about 25 to 60 mol % or having a zeta potential in the range of about +25 to +100 mV in about 0.05 MM KCl solution at about pH 7.5.

8. The therapeutic composition of claim 7 wherein the active ingredient is selected from the group consisting of cyto statics and cytotoxic agents.

9. The therapeutic composition of claim 8, wherein the cytostatics and cytotoxic agents are selected from the group consisting of taxanes, inorganic complexes, mitose inhibitors, hormones, anthracyclines, antibodies, topoisomerase inhibitors, antiinflammtory agents, alkaloids, interleukins, cytokines, growth factors, proteins, peptides, and tetracyclines.

10. A method of selectively targeting a therapeutic composition to a site of angiogenesis in an animal comprising the step of administering to the animal a composition according to claim 7 or 8, and allowing the composition to selectively accumulate to a therapeutically effective level in the vicinity of the site of angiogenesis.

11. The method of claim 5 or 10, wherein the animal is a mammal.

12. The method of claim 10, wherein the composition is administered by a route selected from the group consisting of oral administration, intravenous administration, intramuscular administration, transdermal administration, subcutaneous administration, intraperitoneal administration, intratumoral adminstration, intraarterial administration, instillation and aerosol administration.

13. A method for enhancing the selective association of a composition at an activated vascular site, comprising the step of modifying the composition to have one or more of the characteristics selected from the group consisting of: (a) a zeta potential in the range of about +25 mV to +100 mV in about 0.05 mM KCl solution at about pH 7.5; and (b) an isoelectric point between above 7.5.

14. The method of claim 1, wherein the activated vascular site is indicative of an angiogenesis associated disease.

15. The method of claim 14, wherein the angiogenesis associated disease is selected from the group consisting of diabetic retinopathy, chronic inflammatory diseases, rheumatoid arthritis, dermatitis, psoriasis, stomach ulcers, hematogenous and solid tumors as well as metastases thereof.

16. The method of claim 1, wherein the composition has been modified to increase its zeta potential to a level of at least about +25 mV.

17. The method of claim 1, wherein the composition has been modified through a reaction with a cation forming reagent that increases the isoelectric point of the agent relative to the non-modified agent to a value above 7.5.

18. The method of claim 17, wherein the composition has been modified by reacting with a cation forming reagent selected from the group consisting of ethylene diamine, hexamethylenediamine, triethylene tetraamine, 4-dimethylamino butylamine, N,N-dimethylaminoethyl amine, dimethylamino benzaldehyde, polylysine, and chitosan.

19. The method of claim 17, wherein the composition comprises a peptide or a protein.

20. A therapeutic composition comprising an active ingredient that is therapeutically effective for the treatment of an angiogenesis associated disease, the composition having a zeta potential within a range of about +25 mV to +100 mV in about 0.05 mM KCl solution at about pH 7.5 or an isoelectric point above 7.5, the composition further being labeled or packaged with directions for the administration of the composition to treat an angiogenesis associated disease.

21. A therapeutic composition comprising an active ingredient that is therapeutically effective to inhibit inflammation, the composition having a zeta potential within a range of about +25 to +100 mV in about 0.05 mM KCl solution at about pH 7.5 or an isoelectric point above 7.5, the composition further being labeled or packaged with directions for the administration of the composition to inflammation.

22. A therapeutic composition comprising an active ingredient that is therapeutically effective to promote bone repair or wound healing, the composition having a zeta potential within a range of about +25 to +100 mV in about 0.05 mM KCl solution at about pH 7.5 or an isoelectric point above 7.5, the composition further being labeled or packaged with directions for the administration of the composition to promote bone repair or wound healing.

23. A diagnostic composition comprising an active ingredient that is diagnostically effective for the diagnosis or imaging of an angiogenesis associated disease, the composition having zeta potential within a range of about +25 to +100 mV in about 0.05 mM KCl solution at about pH 7.5 or an isoelectric point above 7.5, the composition further being labeled or packaged with directions for the administration of the composition to diagnose or image an angiogenesis associated disease.

24. The therapeutic composition of claim 7, wherein the active ingredient is selected from the group consisting of etherlipid, alkyllysolecithin, alkyllysophopholipid, lysolipid, alkylphospholipid.

25. The therapeutic composition of claim 24, wherein the etherlipid is selected from the group consisting of 1-O-octadecyl-2-O-methyl-rac-glycero-3-phosphocholine, 1-O-Hexadecyl-2-O-methyl-sn-glycerol, Hexadecyl phosphocholine, Octadecylphosphocholine.

26. The method of claim 1, wherein the composition comprises particles having a zeta potential in the range of about +25 mV to +60 mV in about 0.05 mM KCl solution at about pH 7.5.

27. The method of claim 26, wherein the composition comprises particles having a zeta potential in the range of about +30 to +50 mV in about 0.05 mM KCl solution at about pH 7.5.

28. The imaging composition of claim 3, wherein the composition comprises particles having a zeta potential in the range of about +25 mV to +60 mV in about 0.05 mM KCI solution at about pH 7.5.

29. The composition of claim 28, wherein the composition comprises particles having a zeta potential in the range of about +30 to +50 mV in about 0.05 mM KCl solution at about pH 7.5.

30. The therapeutic composition of claim 7, wherein the composition comprises particles having a zeta potential in the range of about +25 mV to +60 mV in about 0.05 mM KCl solution at about pH 7.5.

31. The composition of claim 30, wherein the composition comprises particles having a zeta potential in the range of about +30 to +50 mV in about 0.05 mM KCl solution at about pH 7.5.

32. The method of claim 12, wherein the composition is modified to increase or decrease its zeta potential to fall within the range of about +25 mV to +60 mV in about 0.05 mM KCl solution at about pH 7.5.

33. A method for identifying an optimal range of zeta potential for a composition for targeting to a specific site comprising evaluating zeta potential for the composition, wherein the composition is associated with different amounts of a cationic component, and identifying an optimal range of zeta potential.

34. A method of modifying a composition to enhance its efficacy comprising the associating of cationic components with the composition to produce a composition having an optimal range of zeta potential.
